(19) United States
(12) Patent Application Publication
(10) Pub. No.: US 2026/0028552 A1
(43) Pub. Date: Jan. 29, 2026

(54) Cationic Surfactants with Improved pH Stability and Their Use

(71) Applicant: Locus Solutions IPCo, LLC, Solon, OH (US)

(72) Inventors: Lee SPEIGHT, Solon, OH (US); Andrew MORRIS, Richmond, VA (US); Daniel HAGAMAN, Mechanicsville, VA (US)

(21) Appl. No.: 18/997,208

(22) PCT Filed: Aug. 4, 2023
(86) PCT No.: PCT/US2023/071706

§ 371 Date: Jan. 21, 2025

Related U.S. Application Data
(60) Provisional application No. 63/395,160, filed on Aug. 4, 2022.
(60) Provisional application No. 63/397,828, filed on Aug. 13, 2022.
(60) Provisional application No. 63/487,860, filed on Mar. 1, 2023.

(51) Int. Cl. C11D 1/52; A01N 47/08; A01N 47/44; A01P 1/00; A61K 8/43; A61K 8/60; A61Q 5/12; B01D 15/36; B01J 47/02; C07C 277/08; C07C 279/12; C07H 1/06; C07H 15/04; C07H 15/10; C11D 3/43; C11D 3/48

(52) U.S. Cl. CPC C11D 1/528; A01N 47/08; A01N 47/44; A01P 1/00; A61K 8/43; A61K 8/602; A61Q 5/12; B01D 15/361; B01J 47/02; C07C 277/08; C07C 279/12; C07H 1/06; C07H 15/04; C07H 15/10; C11D 3/43; C11D 3/48

(57) ABSTRACT

Novel cationic surfactant derivatives are provided, which have enhanced pH stability and surprising ability to clean soils while sanitizing and/or disinfecting in a single application. Methods of their use and production are also provided. Advantageously, the novel surfactants are biodegradable and have reduced toxicity over comparable traditional surfactants.

### CROSS REFERENCE TO RELATED APPLICATION

This application claims priority to U.S. Provisional Patent Application Nos. 63/395,160, filed Aug. 4, 2022; 63/397,828, filed Aug. 13, 2022; and 63/487,860, filed Mar. 1, 2023; each of which is incorporated herein by reference in its entirety.

### BACKGROUND OF THE INVENTION

Consumers utilize, and are exposed to, household and personal care products every day. The daily routine of most consumers includes the use of cleaning and disinfecting products, as well as personal care, cosmetic, and hygiene products. All of these product categories rely on ingredients with surfactancy and/or disinfecting properties, which will be the prominent subject of this invention. Many, if not most, such products contain synthetic chemicals as active ingredients, as well as secondary additives that, for example, help with properties such as viscosity, foaming, corrosion prevention, and solubility of fragrances, dyes, and active components. Unfortunately, too many ingredients found in such products come with a price in terms of human health or environmental risks. This invention provides a new category of biochemical ingredients that reduces these risks across a wide range of critical application categories.

Disinfectants are essential for helping to protect public health. The cleaning and disinfecting industry currently relies almost entirely on functional ingredients, including traditional synthetic chemical ingredients, which are less than ideal in terms of safety and environmental impact. As a society, we must weigh potential health and safety risks against the benefits of protecting human and animal health. This conundrum is due to a paucity of effective ingredients that are also safe for human exposure and the environment. Even with the advent of recent EPA programs, such as the Safer Choice Program, which identifies ingredients with superior environmental and safety profiles, there is still a lack of suitable ingredients that are safe for human exposure and readily biodegrade in the environment.

In particular, virtually all EPA approved active disinfectants (defined as Chemical Pesticides) that are currently used in disinfecting products have various levels of inherent health hazards ranging from skin and eye irritation to the potential for chemical burns, inhalation danger, and/or environmental toxicity. These are the same active ingredients that have been the front line in the public health battle for many years. Without products such as concentrated hypochlorite (bleach), strong acids (formate, hydrochloric acid), organic acids, phenolics, peroxides, and quaternary ammonium compounds (QACs), we would never have achieved the public health improvements of the 20^{th} Century. Further, while each of these well-known chemical pesticides offers highly effective options for broad spectrum disinfection of bacterial, viral, fungal, and parasitic pathogens, each also comes with known risks to human safety and the environment.

Toxicity to humans and domestic animals is the short-term problem with existing personal care, household and disinfecting products. The environmental damage that can be attributed to these ingredients has yet to be fully understood; however, it is largely dose dependent. Short chain alcohols, hypochlorite salts, and peroxides have all been shown to biodegrade with rates that do not suggest significant accumulation in the environment. Large spills or deliberate application of these types of ingredients do cause environmental disruption, though the effects are not long-lasting.

QACs, on the other hand, have been shown to persist in the environment. While biodegradation pathways have been shown under laboratory aerobic conditions, QACs, especially those containing aromatic scaffolds, are prone to accumulate in environmental sludges and partition poorly to aqueous media. This removes QACs from an aerobic environment in which biodegradation can occur. Accumulation of QACs in anaerobic environmental sludges and soils poses a significant challenge. Typical methods to remove other nitrogen-containing environmental contaminants, both natural and human-activity promoted, are severely limited by the presence of QACs.

QAC biodegradation in anaerobic sludge environments is possible, but the principal breakdown products of QACs include short alkyl amines such as methyl amine. Alkyl amines can accumulate within the microbes capable of degrading QACs, leading to inhibition of QAC degrading enzymes and/or toxicity to the microbes themselves. Further complicating the matter, QACs have been shown to inhibit methanogenesis and other anaerobic digestion pathways used by microbes to degrade other compounds. The risk of QACs in the environment thus creates the risk of accumulation of other hazardous chemicals that would typically degrade.

In addition to the problem of QAC accumulation in the environment and its impacts on microbes essential for biodegradation pathways, recent research has suggested that QAC environmental accumulation is accelerating the development of microbes that are resistant to traditional antibiotics. The same genes found to be responsible for conferring resistance to QACs in bacteria are associated with drug resistant bacteria studied by medical researchers. The mechanism of this resistance involves the use of efflux proteins with broad specificity towards exogenous compounds. Thus, accumulation of QACs in the environment may likely lead to the selection of bacteria that are capable of resisting them, and, inadvertently, potentially resist traditional antibiotics.

There are a variety of nature-derived molecules that have been shown to have some efficacy as disinfecting active ingredients. The most studied of these types of molecules are antimicrobial peptides (AMPs), or cationic host defense peptides. While the strong performance and compatibility with human and animal health makes AMPs a prime candidate for use as disinfecting active ingredients, contemporary technology is unable to produce them cost-effectively. The lack of methods to produce AMPs at costs conducive to commercialization has prevented their use as disinfecting active ingredients and as therapeutics.

Ethyl lauroyl arginate (or lauryl arginine ethyl ester, “LAE”) is another biocidal compound of interest in the “green” consumer product industry. LAE is a biodegradable amino acid-based cationic surfactant synthesized from L-arginine, lauric acid and ethanol, which has been approved and generally recognized as safe (GRAS) for some food and biomedical applications by the USA Food and Drug Administration (FDA) and the European Food Safety Agency (EFSA). Nonetheless, depending on the source of the LAE, this compound can exhibit variable and unpredictable surfactant properties. The stability of LAE decreases at higher pH as a result of base-catalyzed hydrolysis, which creates a mixed surfactant system with additional surface-active components that can affect surface tension and critical micelle concentration (CMC) values. (Czakaj et al. 2021). For example, hydrolysis of the ester moiety can yield a zwitterionic species, and/or cause intramolecular cyclization, resulting in the formation of lactam. Thus, high pH instability renders LAE unsuitable for a wide range of alkaline consumer formulations.

The current invention provides improved functionality and safety for a range of personal care products. A representative example in personal care can be seen in hair care products. Shampooing of hair cleans the hair by removing excessive environmental soils and sebum. Shampooing can also result in tangled and unmanageable hair, especially with longer length hair. Furthermore, once the hair dries, it is often left in a dry, rough, staticky, lusterless and/or frizzy condition due to the removal of the hair's natural oils and other natural conditioning and moisturizing components.

To avoid these problems, hair conditioning compositions are typically applied to the hair immediately after shampooing and rinsing. The conditioning composition is worked through the hair and may be then used as a leave-on conditioner or it can be rinsed from the hair with water. Similar types of compositions have also been used in the conditioning of textiles and fibers.

Traditionally, hair and fiber conditioning compositions have utilized cationic surfactants. Cationic surfactants are those in which the surfactant activity resides in the positively charged cation portion of the molecule. The cationic surfactants are therefore attracted to the negatively charged hair or fiber surface and deposited on the hair or fiber. Among cationic surfactants, QACs are particularly suited to the treatment hairs and fibers. Thus, many conditioning products are based on QACs, such as stearyl trimethylammonium chloride, behenyl trimethylammonium chloride and distearyl dimethylammonium chloride.

There are also a variety of nature-derived substances that have been shown to have some efficacy as conditioning ingredients. One such substance is palm oil and its derivatives (e.g., palmitate, glyceryl, stearic acid, sodium laureth sulfate and sodium lauryl sulfate). In shampoos and conditioners, palm oil is used to help restore the natural oils of the hair that are stripped away by other cleansing chemicals present in most shampoos.

Increasingly, consumers are looking for cleaning and disinfecting products, as well as other household and personal care products, that are non-toxic, non-irritating to the skin and/or eyes, and with a reduced impact on the environment. These safer and more sustainable products are still expected to deliver excellent performance on attributes such as cleaning, disinfection, and personal care at parity to current products.

Additionally, different cleaning products are targeted for different types of applications, each with their own characteristic types of soils. Cleaners used on glass and floor surfaces typically need to disperse and solubilize the minerals in hard water in order to avoid streaking. Bath and shower cleaners need to be effective against soap scums and biofilms, which benefit from low pH cleaners in the 3-5 range. Other products used to clean food preparation and storage areas (e.g., kitchen counters and refrigerators) need to be able to wet and remove saturated fats and oils, which benefit from elevated pH levels in the 9-11 range.

Thus, because every type of soil has a different pH that is optimum for cleaning, there is a need for products with functionality across a broad range of product pH levels. Due to the limited set of natural or sustainable materials that meet these needs, formulating safe and environmentally-friendly consumer products remains a challenge.

The current invention addresses needs for improved cleaning, disinfecting, and personal care ingredients that are safer for human exposure and for the environment but do not sacrifice functional attributes across a wide range of applications. This invention provides novel biochemical processes that produce safe, new ingredients having surprising and unexpected attributes comparable to, or better than, existing disinfecting or other ingredients. These novel ingredients offer the very surprising additional benefits of superior stability and functionality across a broader range of pH conditions as compared to current and existing ingredient solutions.

### BRIEF SUMMARY OF THE INVENTION

The present application provides materials and methods for producing functionalized cationic surfactants, including bio-based surfactants and biosurfactants, as well as functionalized cationic surfactants having enhanced properties. More specifically, the subject invention provides surfactant compounds comprising amine-functionalized amino acids, wherein the compounds have pH stability at broad ranges, e.g., pH 2-12. In some instances, a glycolipid scaffold is included to improve biodegradability and in other instances a lauryl carbon chain is used to reduce molecular weight. Methods for producing, formulating and using these compounds are also provided.

Certain cationic amino acid derivative compounds comprising ester moieties, such as lauroyl arginine ethyl ester (LAE or LRE), are unstable at pH 7 or above, resulting in at least two possible undesirable reactions: hydrolysis of the ester, yielding a zwitterionic species, and/or intramolecular cyclization, resulting in the formation of lactam. FIG. 1. These undesirable reactions can negatively impact the structural uniformity and efficacy of the cationic compounds in environments in which neutral to basic pH levels are preferred. Thus, the subject invention provides solutions for overcoming this problem by providing stable cationic surfactant derivative compounds suitable for uses such as, e.g., cleaners, disinfectants, preservatives and conditioners. FIGS. 2-3.

In certain embodiments, the cationic surfactant compounds of the subject invention are functionalized amino alcohols that can be customized based on, for example, the desired functionality, stability, biodegradability and/or its compatibility with other substances. The surfactant compound “XYZ” is preferably an amino acid alcohol having a structure according to General Formula (1):

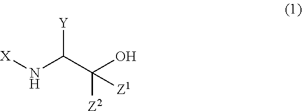

wherein X is a fatty amide derived from a fatty acid or a glycolipid biosurfactant comprising a fatty acid moiety, Y comprises one or more amino acid-derived functional groups, and Z¹ and Z² are independently a hydrogen, an alkyl group (e.g., a methyl) or another substituent, such as a phenyl group or a benzyl group.

More specifically, the X group is preferably derived from a substrate comprising an acyl group with a range of fatty acid carbon chain lengths, e.g., from C2-C22. For example, X can be derived from an acyl halide (e.g., lauroyl chloride) or a glycolipid biosurfactant (e.g., a sophorolipid).

In addition, the Y group preferably comprises one or more amino acid-derived functional groups (e.g., arginine, lysine and/or histidine), which can be selected based on the desired functionality of the surfactant compound.

Finally, a reducing agent can be used to improve the pH stability of the compound by, e.g., reducing any ester groups to a primary, secondary or tertiary alcohol.

In certain embodiments, the cationic compounds of the subject invention comprise surfactant molecules comprising amine-functionalized amino alcohols. FIG. 4. These molecules include, for example, lauryl amino alcohols, and glycolipid derivatives, such as linear sophorolipid amino alcohol amides.

In certain exemplary embodiments, the compounds can have one of the following structures:

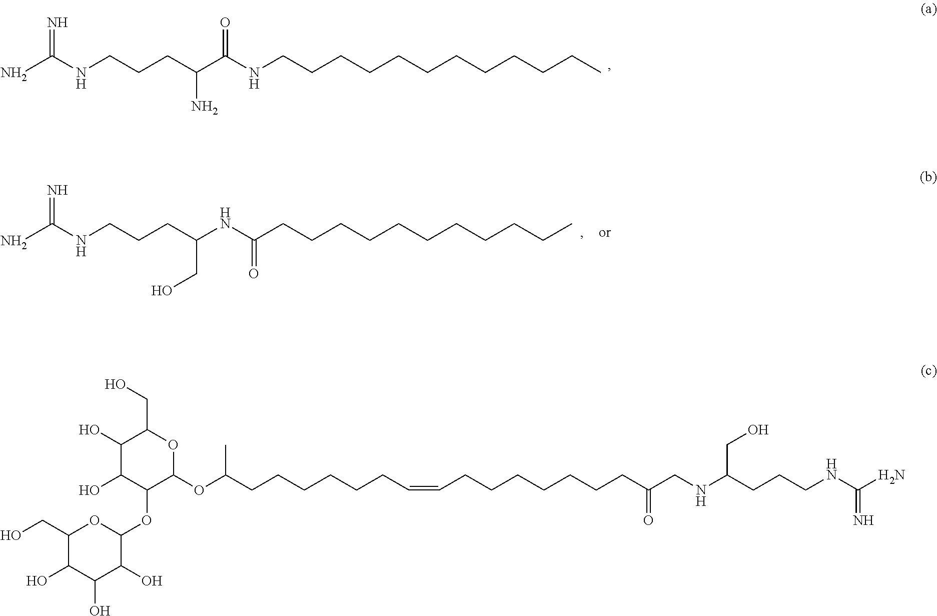 [SMILES: CCCCCCCCCCCCNC(=O)C(N)CCCNC(=N)N ; CCCCCCCCCCCC(=O)NC(CO)CCCNC(=N)N ; CC(CCCCCC/C=C\CCCCCCCC(=O)CNC(CO)CCCNC(=N)N)OC1OC(CO)C(O)C(O)C1OC1OC(CO)C(O)C(O)C1O]

wherein structure (a) is a lauryl arginine amide; structure (b) is a lauryl argininol alcohol; and structure (c) is a linear sophorolipid argininol amide.

The subject invention further provides a method for producing a derivatized surfactant compound (XYZ) having improved pH stability, which comprises coupling a biosurfactant or other fatty acid-containing substrate (X′) with an amide (Y′) comprising one or more amino acid functional groups (Y) to produce an amino acid-functionalized surfactant (XY). Reagent Z′ is then used to reduce any desired ester groups to a primary, secondary or tertiary alcohol, either before, during or after coupling of XY, in order to produce XYZ. FIGS. 5-10.

The subject invention provides advantageous novel compounds, including, for example, those described in the Figures and throughout the subject description, as well as formulations comprising these novel compounds and methods of their use. Advantageously, the cationic surfactant compounds produced and/or derivatized according to the subject invention can withstand the negative reactive influences present in neutral to basic pH environments that result in instability and loss of efficacy, e.g., ester hydrolysis and/or lactam formation. Furthermore, they can be used as active ingredients in households, industrial settings, office and retail settings, in personal care, and in healthcare as more “green” alternatives to compounds such as QACs.

### BRIEF DESCRIPTION OF THE FIGURES

FIG. 1 depicts a reaction scheme in which amine-derivatized amino acid compounds comprising ester moieties are hydrolyzed to produce a zwitterionic species or converted to lactam at pH 7 or above.

FIG. 2 depicts a reaction scheme according to an embodiment of the subject invention showing the general synthetic stabilization of any amino acid or amino acid ester via conversion to an amino alcohol.

FIGS. 3A-3B depict a comparison of pH stability between lauryl arginine ethyl ester (LRE) and a cationic amino alcohol surfactant according to an embodiment of the subject invention (LRO) in different conditions of incubation at pH 7 (A) and pH 10 (B). Oven=45° C., 50% relative humidity; ambient=room temperature.

FIG. 4 depicts exemplary cationic amino alcohol surfactant derivatives with a primary amine connection to R₁=lauryl carbon chain (1) or a linear sophorolipid scaffold (2-3). R₂=an amino acid-derived functional group.

FIG. 5 depicts reaction schemes for synthesis of amino alcohols, as exemplified by synthesis of argininol.

FIG. 6 depicts a reaction scheme for the synthesis of a lauryl arginine amide derivative through amide coupling.

FIG. 7 depicts a reaction scheme for the synthesis of a lauryl argininol amide derivative through amide coupling.

FIG. 8 depicts a reaction scheme according to an embodiment of the subject invention showing hydrolysis of a di-acetylated lactonic sophorolipid to produce a linear sophorolipid.

FIG. 9 depicts a reaction scheme according to an embodiment of the subject invention showing amide coupling of a linear SLP substrate to produce a long-chain amide containing a cationic amino acid-derived functional group.

FIGS. 10A-10B depict (A) a reaction scheme according to an embodiment of the subject invention showing oxidative cleavage of a linear SLP substrate to produce a truncated acid, and (B) a reaction scheme according to an embodiment of the subject invention showing amide coupling of the truncated acid to produce a short-chain amide containing a cationic amino acid-derived functional group.

### DETAILED DESCRIPTION

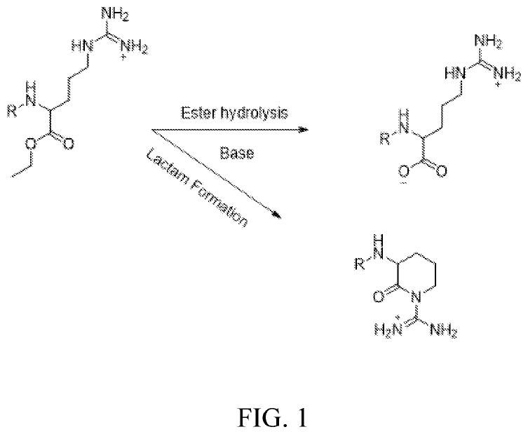

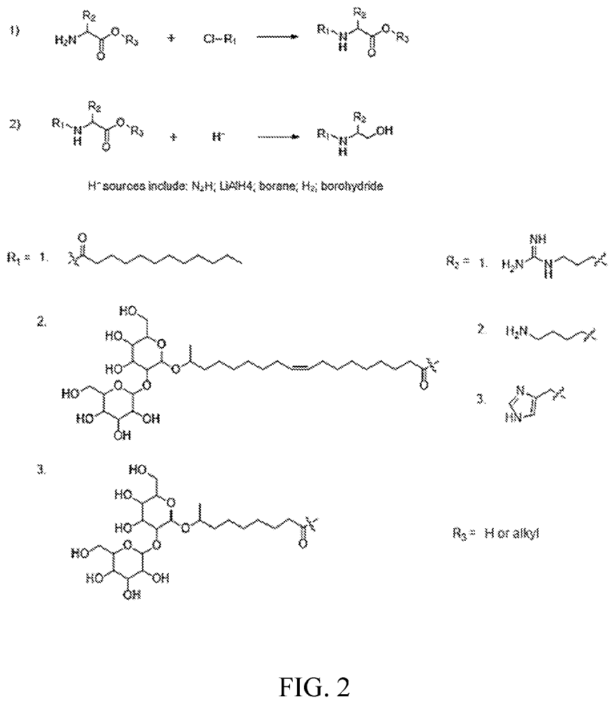

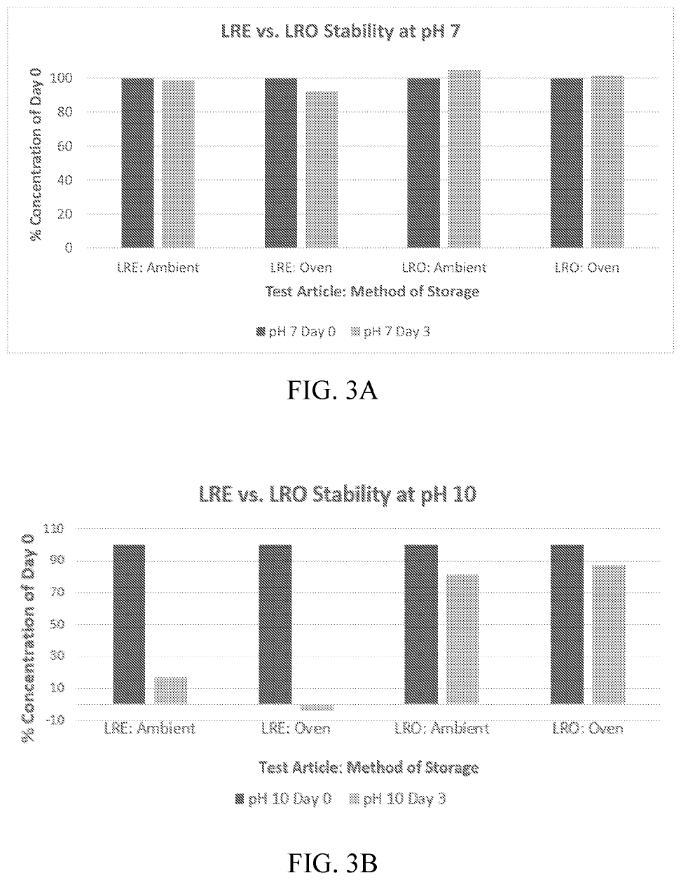

Certain cationic surfactant molecules, such as, for example, LAE (or LRE), contain problematic ester moieties that render the molecules unsuitable for environments of pH 7 or greater. At these pH levels, undesirable reactions result in loss of efficacy as, e.g., disinfecting active ingredients. FIG. 1. The subject invention provides compounds that, advantageously, have excellent activity yet are stable across a broad range of pH, thereby providing more predictable and broad-range pH applicability for a variety of surfactants comprising cationic amino acid-derived functional groups. See FIGS. 2-3.

### Selected Definitions

As used herein, a “green” compound or material means that it is at least 95% derived from natural, biological and/or renewable sources, such as plants, animals and/or microorganisms, and furthermore, the compound or material is biodegradable. Additionally, in some embodiments, “green” compounds or materials are minimally toxic to humans and can have a LD50>5000 mg/kg. A “green” product preferably does not contain any of the following: non-plant based ethoxylated surfactants, linear alkylbenzene sulfonates (LAS), ether sulfates surfactants or nonylphenol ethoxylate (NPE). In certain preferred embodiments, the cationic derivative molecules described herein are “green” compounds with minimal toxicity to users.

As used herein, a “derivative” is a substance that is created from another substance via a chemical reaction, for example, by exchanging one atom or a group of atoms in the parent substance with another atom or a group of atoms.

As used herein, a “scaffold” of a molecule means the core structure of the molecule to which functional groups are attached.

As used herein, “surfactant” refers to a substance or compound that reduces surface tension when dissolved in water or water solutions, or that reduces interfacial tension between liquids, solids and/or gases. The term “surfactant” thus includes cationic, anionic, nonionic, zwitterionic, amphoteric agents and/or combinations thereof. By “biosurfactant” is meant a surfactant produced by a living cell and/or using naturally-derived substrates.

The properties of a surface-active molecule can be measured by hydrophile-lipophile balance (HLB). HLB is the balance of the size and strength of the hydrophilic and lipophilic moieties of a surface-active molecule. Specific HLB values are required to, for example, form a stable emulsion. In water/oil and oil/water emulsions, the polar moiety of the surface-active molecule orients towards the water, and the non-polar group orients towards the oil, thus lowering the interfacial tension between the oil and water phases.

HLB values range from 0 to about 20, with lower HLB (e.g., 10 or less) being more oil-soluble and suitable for water-in-oil emulsions, and higher HLB (e.g., 10 or more) being more water-soluble and suitable for oil-in-water emulsions. Other properties, such as foaming, wetting, detergency and solubilizing capabilities, are also dependent upon HLB.

As used herein, “base surfactant” refers to a surfactant or amphiphilic molecule that exhibits a strong tendency to adsorb at interfaces in a relatively ordered fashion, oriented perpendicular to the interface.

As used herein, the term “syndetic” (meaning to join or link together, as in mixing water and oil) refers to a relatively weak amphiphile that exhibits a significant ability to adsorb at an oil-water interface (from either the water phase, hence a “hydrophilic syndetic,” or from the oil phase, hence a “hydrophobic syndetic”) only when the interface already bears an adsorbed layer of a base surfactant or mixture of base surfactants. Adsorption of syndetics at oil-water interfaces is thought to affect the spacing and/or the order of the adsorbed ordinary surfactants in a manner that is highly beneficial to the production of very low oil-water interfacial tensions, which in turn increases the solubilization of oils and/or the removal of oils from solid materials and/or surfaces.

As used herein, an “isolated” or “purified” nucleic acid molecule, polynucleotide, polypeptide, protein or organic compound, such as a small molecule, is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of other molecules, or the amino acids that flank it, in its naturally-occurring state. An “isolated” strain means that the strain is removed from the environment in which it exists in nature. Thus, the isolated strain may exist as, for example, a biologically pure culture, or as spores (or other forms of the strain).

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduces” means a negative alteration, and “increases” means a positive alteration, wherein the alteration is at least 0.001%, 0.01%, 0.1%, 1%, 5%, 10%, 15%, 20%, 25%, 30%, 35%, 45%, 50%, 55%, 60%, 65%, 70%, 75%, 80%, 85%, 90%, 95%, 99%, or 100%, inclusive of all values therebetween.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference.

### Cationic Surfactant Molecules

The subject invention provides novel cationic surfactant molecules having broad pH stability, e.g., pH 2-12. In certain embodiments, the molecules are functionalized amino alcohols that can be customized based on, for example, the desired functionality, stability, biodegradability and/or its compatibility with other substances. The subject surfactant compounds, “XYZ,” are preferably amino acid-derived alcohols having a structure according to General Formula (1):

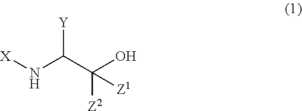

wherein X is a fatty amide derived from a fatty acid or a glycolipid biosurfactant comprising a fatty acid moiety, Y comprises one or more amino acid-derived functional groups, and Z¹ and Z² are independently a hydrogen, an alkyl group (e.g., a methyl) or another substituent, such as a phenyl groups or a benzyl group. In certain preferred embodiments, Z¹ and Z² are hydrogen, leading to improved atom economy; however, the use of alkyl groups, though increasing the carbon content and reducing atom economy, can reduce the HLB of the molecule if such properties are desirable.

The X group is preferably derived from a substrate comprising an acyl group with a range of fatty acid carbon chain lengths, e.g., from C2-C22. For example, X can be derived from an acyl halide (e.g., lauroyl chloride) or a biosurfactant (preferably a glycolipid, e.g., a sophorolipid). In certain embodiments, the selection of X can be based on a desired surfactant property, for example, a longer carbon chain length can lower the critical micelle concentration (CMC) of the molecule.

In addition, the Y group preferably comprises one or more amino acid-derived functional groups (e.g., arginine, lysine and/or histidine), which can also be selected based on the desired functionality of the surfactant compound, for example, antimicrobial properties.

Finally, a nucleophilic reducing agent can be used to improve the pH stability of the compound by, e.g., reducing any ester groups to a primary, secondary or tertiary alcohol.

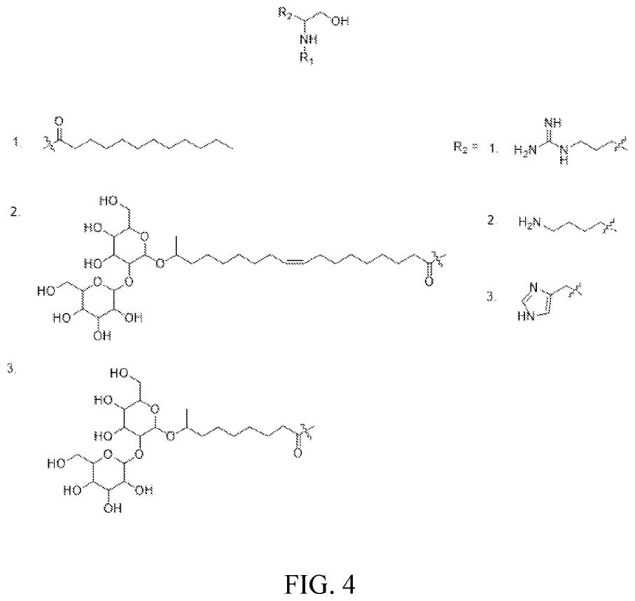

In certain embodiments, the cationic derivative compounds of the subject invention comprise surfactant molecules comprising amine-functionalized amino alcohols. FIG. 4. These molecules include, for example, lauryl amino alcohols, and glycolipid derivatives, such as linear sophorolipid amino alcohol amides.

In exemplary embodiments, the cationic molecules according to the subject invention have the following derivative structures. The present invention pertains to the compounds represented by the General Formulas (2) and (3), as well as salts and hydrates thereof, geometric and optical isomers thereof, and polymorphic forms thereof.

In certain embodiments, the cationic surfactant derivative is an amino acid alcohol having a structure according to General Formula (2):

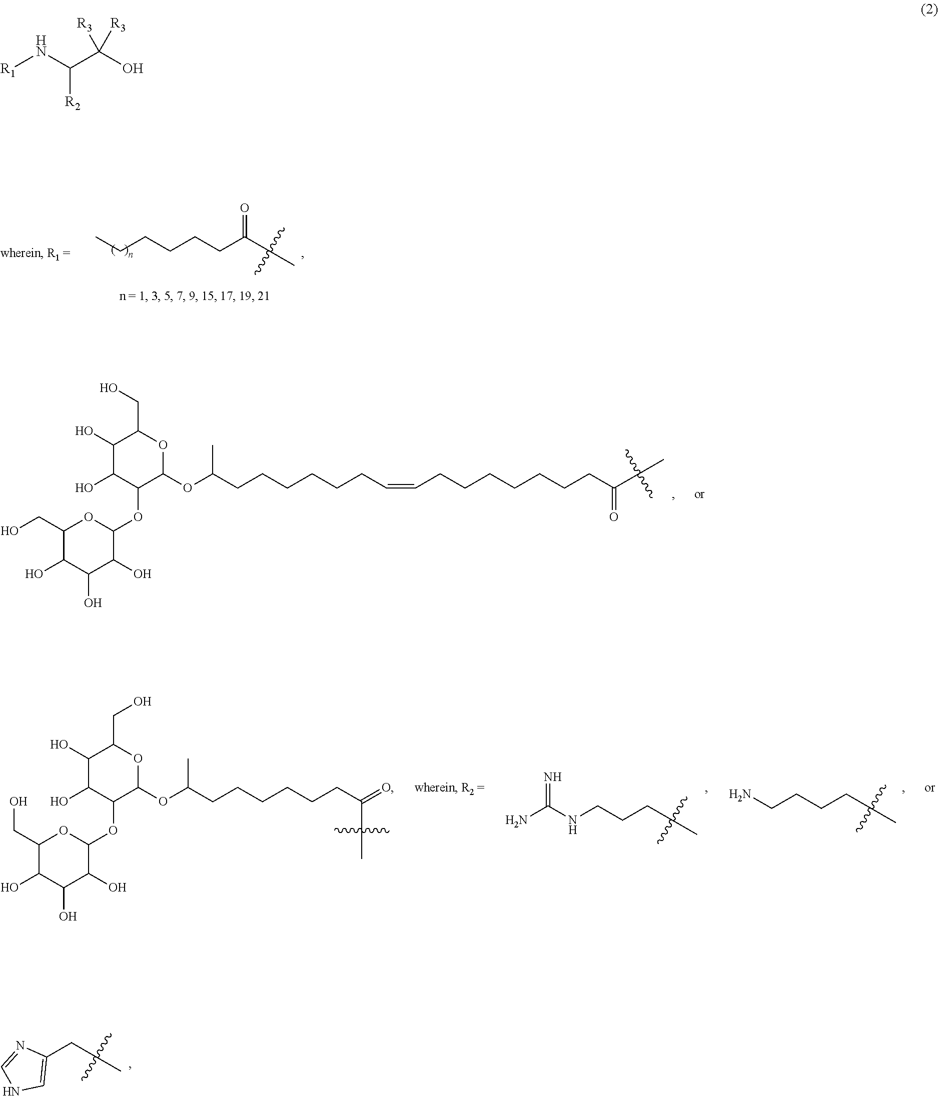

and
wherein R₃=H, Me, Et, MePh, Bu, sec-Bu or t-Bu.

In certain embodiments, the cationic surfactant derivative has a structure according to General Formula (3):

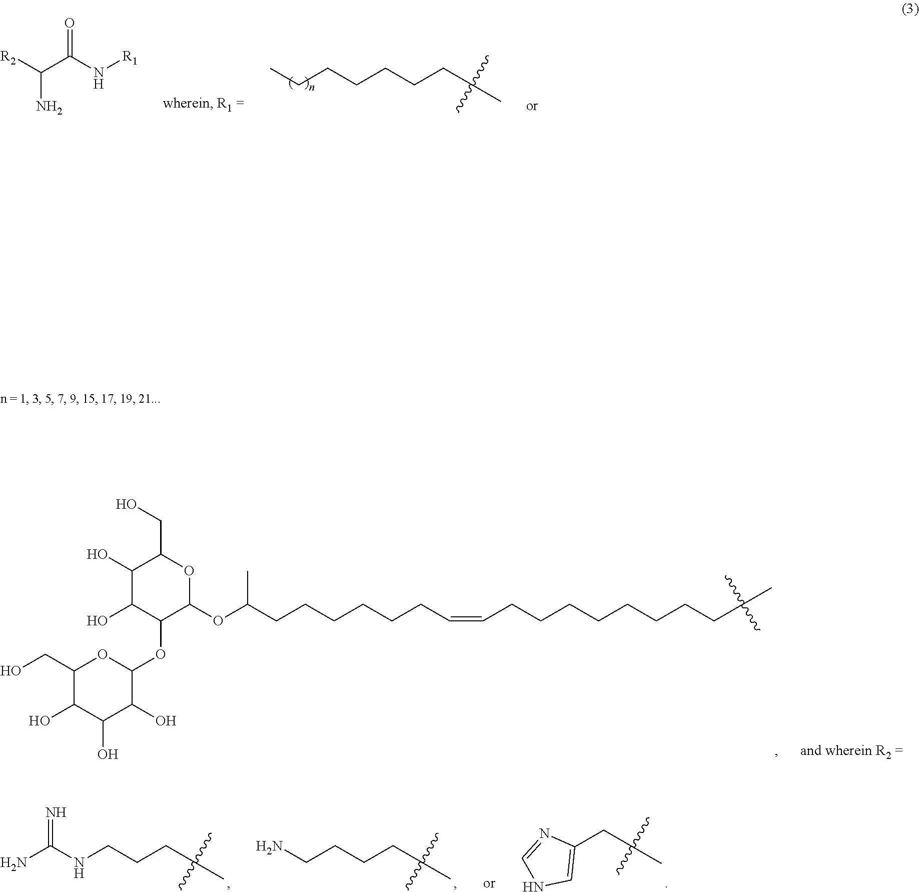

In some embodiments, R₁ in General Formulas (2) and/or (3) is an aliphatic acyl group derived from a C2-C22 acyl halide (or acid halide). Acyl halides can include, but are not limited to, lauroyl, octanoyl, decanoyl, dodecanoyl, myristyroyl, palmitoyl, and behenoyl halides, wherein the halide is fluoride, chloride, bromide or iodide. In preferred embodiments, the acyl halide is an acyl chloride.

In some embodiments, R₁ in General Formulas (2) and/or (3) is a biosurfactant acyl moiety, preferably a glycolipid scaffold.

Glycolipids can include, for example, sophorolipids (SLP), rhamnolipids (RLP), cellobiose lipids, trehalose lipids and/or mannosylerythritol lipids (MEL). Other biosurfactants can include, for example, lipopeptides, such as, surfactin, iturin, fengycin, arthrofactin, amphisin, viscosin, lichenysin, paenibacterin, polymyxin and/or battacin, or another type of amphiphilic molecule, such as, for example, fatty acids, saponins, cardiolipins, pullulan, emulsan, lipomanan, alasan, and/or liposan. In some embodiments, the biosurfactants are produced as a result of fermentation of a biosurfactant-producing organism and/or are derived from naturally-occurring substrate materials.

In some embodiments, R₂ in General Formulas (2) and/or (3) is any amino acid-derived functional group, e.g., an amino acid side chain, including those of aliphatic, aromatic, acidic, basic, hydroxylic, sulfur-containing and/or amidic amino acids. Amino acids according to the subject invention include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, salts and hydrates thereof, geometric and optical isomers thereof, and polymorphic forms thereof.

In certain exemplary embodiments, the compounds of the subject invention have one of the following structures:

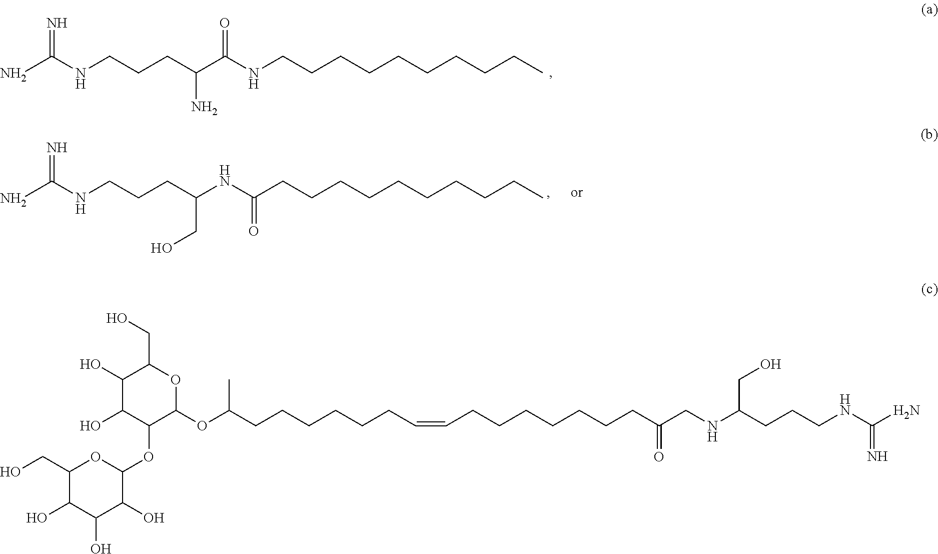 [SMILES: CCCCCCCCCCNC(=O)C(N)CCCNC(=N)N ; CCCCCCCCCCC(=O)NC(CO)CCCNC(=N)N ; CC(CCCCCC/C=C\CCCCCCCC(=O)CNC(CO)CCCNC(=N)N)OC1OC(CO)C(O)C(O)C1OC1OC(CO)C(O)C(O)C1O]

wherein structure (a) is a lauryl arginine amide; structure (b) is a lauryl argininol alcohol; and structure (c) is a linear sophorolipid argininol amide.

Methods of producing these compounds are also provided, wherein the methods generally comprise coupling a surfactant R-group (R₁) or a proton to the C-terminus of an amine-functionalized amino acid or by reducing the carboxylic acid of an amino acid to an alcohol (producing an amino alcohol) and coupling the surfactant R-group or proton with the N-terminus of the amino alcohol. In certain preferred embodiments, the amino alcohol is argininol.

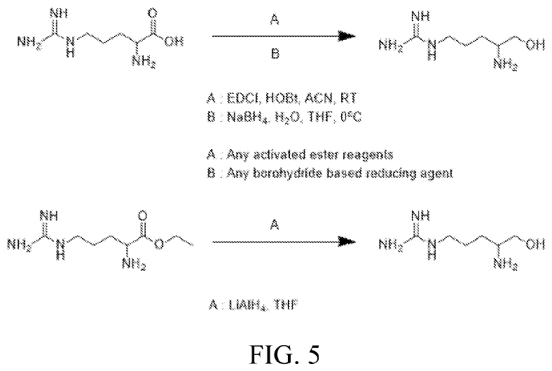

As shown in FIG. 5, synthesis of amino alcohols can be carried out for amine-derivatized amino acids with ester moieties comprising different R′ groups at the ester oxygen, e.g., R′=H, or an alkyl group. The ester moiety can be converted into a hydroxy group via reaction with any activated ester reagent at room temperature, such as, for example, 1-Ethyl-3-(3-dimethylaminopropyl) carbodiimide (EDCl), hydroxybenzotriazole (HOBt), and/or acetonitrile (ACN); and reaction with any borohydride based reducing agent at about 0-10° C., for example, sodium cyanoborohydride, sodium triacetoxyborohydride or sodium borohydride, in a reaction medium comprising tetrahydrofuran (THF) and/or water. In some embodiments, e.g., when R′ is an alkyl group, the ester moiety can be reduced directly using a reducing agent such as, e.g., lithium aluminum hydride (LiAlH₄) in THF.

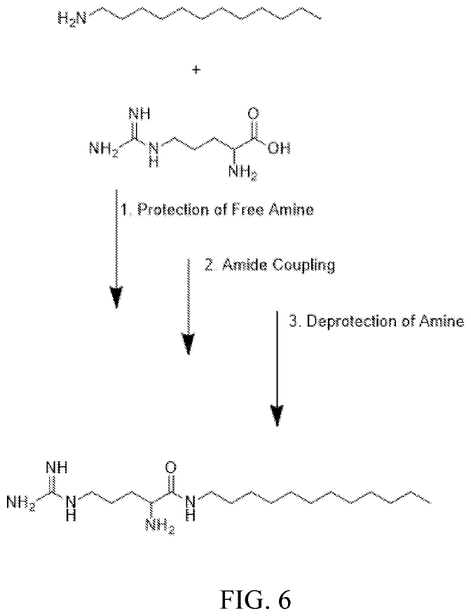

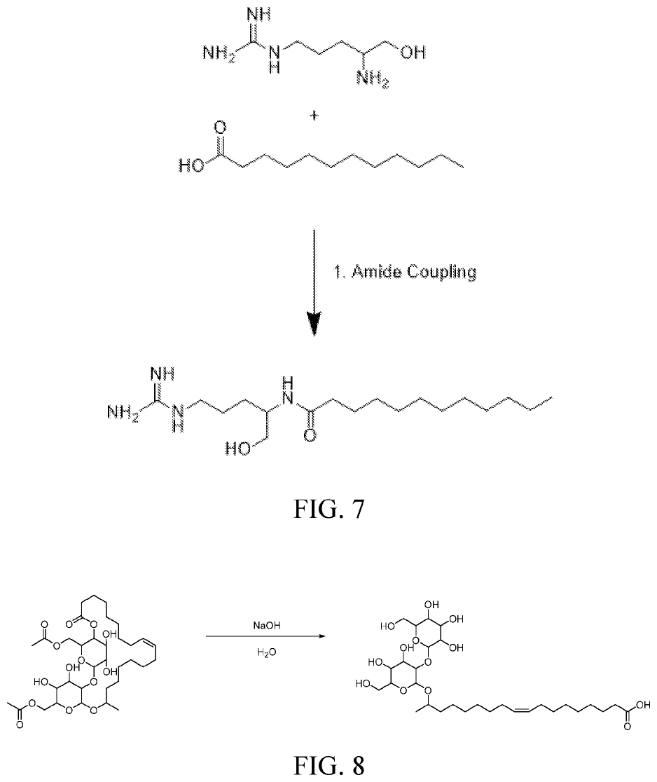

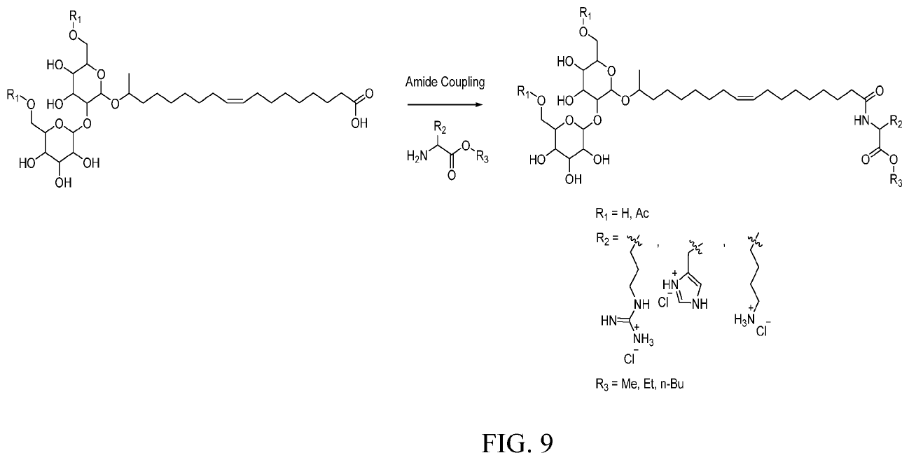

As shown in FIGS. 6-7, coupling to an amine-derivatized amino acid, or to an amino alcohol, can be carried out using amide coupling, for example, as provided herein. See also, FIG. 9.

In certain embodiments, to further functionalize the molecule(s), the surfactant derivatives according to the subject invention can be subjected to alkoxylation and/or sulfur oxidation at, e.g., the side-chain alcohol.

Advantageously, the compounds according to the subject invention are biodegradable, have broad pH stability, and broad spectrum antimicrobial properties. Furthermore, when compared to prior art surfactant derivatives, such as those described in US Patent Pub. No. 2021/0284604 A1, the subject invention provides superior compounds due to, for example, the reduction of the structures' backbone carbonyl group (versus an electrophilic oxidized carbon), the preference for adding H₂ to the base structure while others add multiple carbon atoms (leading to lower atom economy), and a novel alcohol functionality that improves water solubility and facilitates alkoxylation for increased functionalization.

### Cationic Linear Sophorolipid Derivatives

In some embodiments, the cationic surfactant molecules of the subject invention comprise sophorolipid (SLP) scaffolds. Sophorolipids are glycolipid biosurfactants produced by, for example, various yeasts of the Starmerella clade. SLP consist of a disaccharide sophorose linked to long-chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid can have, for example, 11 to 20 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). In most cases, fermentation of SLP results in a mixture of hydrophobic (water-insoluble) SLP, including, e.g., lactonic SLP, mono-acetylated linear SLP and di-acetylated linear SLP, and hydrophilic (water-soluble) SLP, including, e.g., non-acetylated linear SLP.

As used herein, the term “sophorolipid,” “sophorolipid molecule,” “SLP” or “SLP molecule” includes all forms, and isomers thereof, of SLP molecules, including, for example, acidic (linear) SLP and lactonic SLP. Further included are mono-acetylated SLP, di-acetylated SLP, esterified SLP, SLP with varying hydrophobic chain lengths, SLP with fatty acid-amino acid complexes attached, and other, including those that are and/or are not described within in this disclosure.

In preferred embodiments, the SLP according to the subject invention are represented by General Formula (4) and/or General Formula (5), and are obtained as a collection of multiple types of structural homologues:

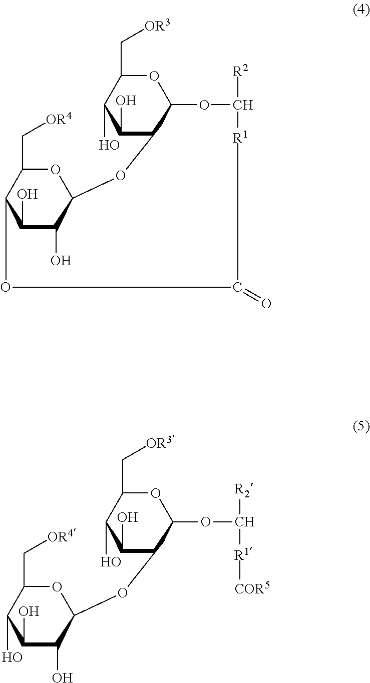

where R¹ and R^{1′} independently represent saturated hydrocarbon chains or single or multiple, in particular single, unsaturated hydrocarbon chains having 8 to 20 carbon atoms, which can be linear or branched and can comprise one or more hydroxy groups, R² and R^{2′} independently represent a hydrogen atom or a saturated alkyl functional group or a single or multiple, in particular single, unsaturated alkyl functional group having 1 to 9 carbon atoms, more preferably 1 to 4 carbon atoms, which can be linear or branched and can comprise one or more hydroxy groups, and R³, R^{3′}, R⁴ and R^{4′} independently represent a hydrogen atom or —COCH₃.

The subject invention provides materials and methods for producing, derivatizing and purifying sophorolipids (SLP). Advantageously, the subject invention is suitable for industrial scale production of purified SLP derivatives and uses safe and environmentally-friendly, or “green,” materials and processes.

In certain embodiments, the subject invention provides cationic SLP derivative molecules, including those that are described in the Figures and throughout the subject Description. In certain embodiments, the cationic SLP derivative molecules are produced according to the methods described herein.

Advantageously, the cationic SLP surfactants of the subject invention have a novel mechanism for wetting and solubilizing anionic soils, such as long chain polysaccharides (e.g., pectin) and/or complex glycoproteins (e.g., casein and mucin). This is due to the ability of the cationic moiety to bind strongly to the anionic backbone of such soils, while the polar sophorose group drives solubilization of the entire complex. Especially at elevated pH levels, anionic sugars and glycoproteins have a negative zeta potential. Binding of a cation to those negative charges can destabilize and cause precipitation without the presence of a nonionic, but strongly hydrophilic, functional group, such as what the sophorose provides.

### Production of Standardized SLP Molecular “Substrates”

In some embodiments, the subject methods initially comprise producing standardized SLP molecular “substrates” for producing derivatized and/or purified SLP. In certain embodiments, this entails cultivating a sophorolipid-producing yeast in a submerged fermentation reactor comprising a tailored oleochemical feedstock to produce a yeast culture product, said yeast culture product comprising fermentation broth, yeast cells and SLP having a mixture of two or more molecular structures.

The mixture of molecular structures can comprise, for example, lactonic SLP, linear SLP, de-acetylated SLP, mono-acetylated SLP, di-acetylated SLP, esterified SLP, SLP with varying hydrophobic chain lengths, SLP with fatty acid-amino acid complexes attached, and others, including those that are and/or are not described within in this disclosure.

In certain embodiments, the distribution of the mixture of SLP molecules can be altered by adjusting fermentation parameters, such as, for example, feedstock, fermentation time, and dissolved oxygen levels.

As used herein “fermentation” refers to growth or cultivation of cells under controlled conditions. The growth could be aerobic or anaerobic. Unless the context requires otherwise, the phrase is intended to encompass both the growth phase and product biosynthesis phase of the process.

As used herein, a “broth,” “culture broth,” or “fermentation broth” refers to a culture medium comprising at least nutrients. If the broth is referred to after a fermentation process, the broth may comprise microbial growth byproducts and/or microbial cells as well.

The microbe growth vessel used according to the subject invention can be any fermenter or cultivation reactor for industrial use. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

In one embodiment, the fermentation reactor may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, SLP concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

The microorganisms utilized according to the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microorganism is a yeast or fungus. Examples of yeast and fungus species suitable for use according to the current invention, include, but are not limited to Starmerella spp. yeasts and/or Candida spp. yeasts, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

In certain embodiments, the cultivation method utilizes submerged fermentation in a liquid growth medium comprising a tailored oleochemical feedstock.

In one embodiment, the liquid growth medium comprises one or more sources of carbon. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, madhuca oil, soybean oil, rice bran oil, olive oil, corn oil, sunflower oil, sesame oil, and/or linseed oil; powdered molasses, etc. These carbon sources may be used independently or in a combination of two or more.

In preferred embodiments, the fermentation medium comprises dextrose. In another preferred embodiment, the oleochemical feedstock is tailored to include a source of oleic acid. In certain embodiments, the oleic acid content is high, e.g., at least 75%, at least 80%, at least 85%, at least 90%, at least 95%, or at least 99%. In some embodiments, the oleochemical feedstock comprises oleic acid sources exclusively.

Examples of oleic acid sources include, but are not limited to, high oleic soybean oil, high oleic sunflower oil, high oleic canola oil, olive oil, pecan oil, peanut oil, macadamia oil, grapeseed oil, sesame oil, poppyseed oil, pure oleic acid, madhuca oil, oleic acid alkyl esters, and/or triglycerides of oleic acid. In preferred embodiments, high oleic soybean oil, pure oleic acid, and/or oleic acid alkyl esters are used.

Advantageously, in certain embodiments, use of high-oleic acid and/or exclusively-oleic acid oleochemical feedstock results in a yeast culture product comprising a narrower diversity of SLP molecular structures than with feedstocks containing sources of other fatty acids, wherein the principal SLP molecules produced contain a C18 carbon chain and a single unsaturated bond at the ninth carbon. For example, in certain embodiments, greater than 50% of the SLP molecules contain an C18 carbon chain, preferably greater than 70%, more preferably greater than 85%.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low oxygen-containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

In certain embodiments, the dissolved oxygen (DO) levels are controlled during fermentation to narrow the structural diversity of SLP molecules produced in the yeast culture product. Preferably, the DO levels are maintained at high levels such that, for example, oxygen transfer occurs at a rate at or above 50 mM, at or above 55 mM, at or above 60 mM, at or above 65 mM, or at or above 70 mM per liter per hour.

In some embodiments, the method for cultivation may further comprise adding additional acids and/or antimicrobials in the liquid medium before and/or during the cultivation process. Antimicrobial agents or antibiotics (e.g., streptomycin, oxytetracycline) are used for protecting the culture against contamination. In some embodiments, however, the metabolites produced by the yeast culture provide sufficient antimicrobial effects to prevent contamination of the culture.

In one embodiment, prior to inoculation of the reactor, the components of the liquid culture medium can optionally be sterilized. In one embodiment, sterilization of the liquid growth medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v).

In one embodiment, the equipment used for cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Gaskets, openings, tubing and other equipment parts can be sprayed with, for example, isopropyl alcohol. Air can be sterilized by methods know in the art. For example, the ambient air can pass through at least one filter before being introduced into the vessel. In other embodiments, the medium may be pasteurized or, optionally, no heat at all added, where the use of pH and/or low water activity may be exploited to control unwanted microbial growth.

The pH of the culture should be suitable for the microorganism of interest. In some embodiments, the pH is about 2.0 to about 7.0, about 3.0 to about 5.5, about 3.25 to about 4.0, or about 3.5. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed.

In one embodiment, the method of cultivation is carried out at about 5° to about 100° C., about 15° to about 60° C., about 20° to about 45° C., about 22° to about 35° C., or about 24° to about 28° C. In one embodiment, the cultivation may be carried out continuously at a constant temperature. In another embodiment, the cultivation may be subject to changing temperatures.

According to the subject methods, the microorganisms can be cultivated in the fermentation system for a time period sufficient to achieve a desired effect, e.g., production of a desired amount of cell biomass or a desired amount of SLP. The microbial growth by-product(s) produced by microorganisms may be retained in the microorganisms and/or secreted into the growth medium. The biomass content may be, for example from 5 g/l to 180 g/l or more, from 10 g/l to 150 g/l, or from 20 g/l to 100 g/l.

In certain embodiments, fermentation of the yeast culture occurs for about 40 to 150 hours, or about 48 to 140 hours, or about 72 to 130 hours or about 96 to 120 hours. In certain specific embodiments, fermentation time ranges from 48 to 72 hours, or from 96 to 120 hours.

In some embodiments, the fermentation cycle is ended once the dextrose and/or oleic acid concentrations in the medium are exhausted (e.g., at a level of 0% to 0.5%). In some embodiments, the end of the fermentation cycle is determined to be a time point when the microorganisms have begun to consume trace amounts of SLP.

In certain embodiments, production of the SLP molecular “substrate” further comprises post-fermentation alteration of the SLP molecules produced in the yeast culture product. In one embodiment, this crude SLP composition is hydrolyzed to produce linear SLP. In some embodiments, the linear SLP are de-acetylated. In some embodiments, the linear SLP are peracetylated.

In some embodiments, the method comprises subjecting the crude SLP to alkaline hydrolysis. For example, in one embodiment, the crude SLP can be mixed with equimolar to 1.5 molar concentrations of a base solution, such as, for example, a solution of sodium hydroxide, potassium hydroxide, and/or ammonium hydroxide, to adjust the pH to, e.g., about 4 to 11, about 5 to 11, about 6 to 12, or preferably, about 7 to 9. In certain embodiments, this is achieved by treating the crude SLP with the hydroxide salt solution for 2 to 24 hours, 3 to 20 hours, or 4 to 16 hours at an elevated temperature of, e.g., 75 to 100° C., 80 to 95° C., or 85 to 90° C.

According to the subject methods, the hydrolysis process results in breakage of the lactone bond of lactonic SLP and conversion thereof to a crude linear SLP. FIG. 8. In certain embodiments, a portion of the crude linear SLP are acetylated, di-acetylated, or peracetylated, wherein the portion comprises from, for example, 1% to 100%, 5% to 75%, or 10 to 50% of the total amount of SLP molecules. In another embodiment, a mono- or di-acetylated SLP molecule can be de-acetylated via the same alkaline hydrolysis process.

In some embodiments, when spectator cations are or may be present in the hydrolysis process, the crude linear SLP are purified using cation exchange resins. More specifically, in preferred embodiments, the crude linear sophorolipids are circulated through an ion exchange bed containing cation exchange sites using, for example, a peristaltic pump or other type of pump, for a period of time from, e.g., 15 minutes to 20 hours, 3 hours to 15 hours, 4 hours to 12 hours, or preferably, 30 minutes to 3 hours.

The amount of cation exchange sites can be, for example, equimolar to 1.5 molar the concentration of hydroxide salts used for the alkaline hydrolysis.

Advantageously, the ion exchange resins provide novel methods for purifying SLP molecules, as well as novel methods for neutralizing the pH of a reaction product without the need for standard quenching methods, which can dilute and/or change the chemical make-up of an end product.

In preferred embodiments, the linear SLP, having spectator cations removed, serve as the standardized substrates for one or more derivatization and/or purification reactions.

### Obtaining Derivatized SLP Containing a Short-Chain or Long-Chain Amide Functional Group

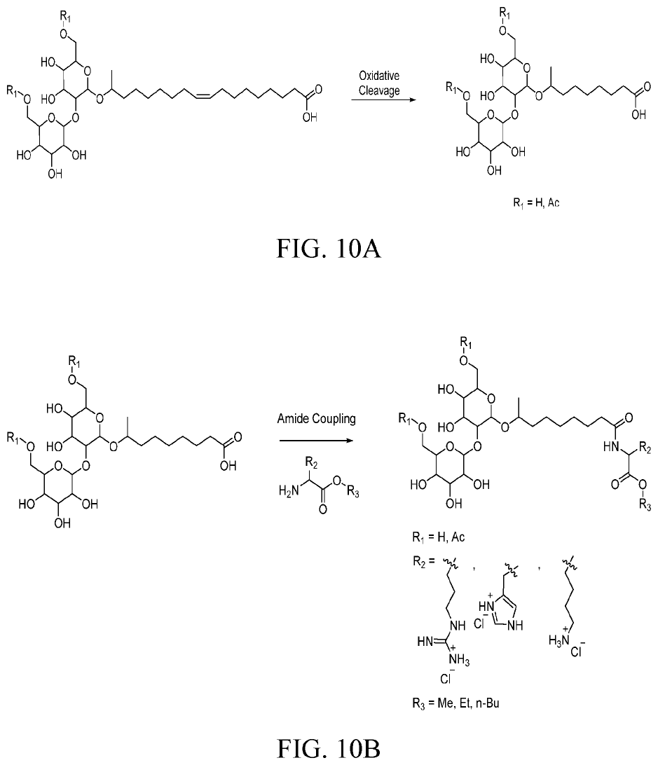

In certain embodiments, the linear SLP substrate can be installed with an amide comprising cationic amino acid functional groups to produce a long-chain amide derivative (e.g., C18). FIG. 9. In some embodiments, the linear SLP substrate can be converted to a short-chain amide (e.g., C9) by first, truncating the fatty acid tail via oxidative cleavage, and second, installing an amide comprising cationic amino acid functional groups to the truncated acid. FIGS. 10A-10B.

Coupling agents for use in amide installation according to the subject invention can include, for example, 1-Ethyl-3-(3-dimethylaminopropyl) carbodiimide (EDCl/HOBt), Benzotriazol-1-yloxytripyrrolidinophosphonium hexafluorophosphate (PYBOP), 2-(1H-Benotriazole-1-yl)-1,1,3,3-tetramethylaminium tetrafluoroborate (TBTU), and/or N,N′-Dicyclohexylcarbodiimide/1-Hydroxybenzotriazole (DCC/HOBt). In certain embodiments, the preferred coupling agent is EDCl/HOBt.

### Ion Exchange/Purification

In certain embodiments, the unique cationic nature of the SLP derivatives of the subject invention allows for cationic ion exchange resins to be used for selective purification, e.g., selective retention of cationic species and/or selective removal of unreacted SLP and SLP that did not contain the desired carbon chain length or character. Thus, in certain embodiments, the subject invention provides novel methods of purifying SLP and SLP derivatives using cationic ion exchange resins.

In certain embodiments, following installation of the cationic cargo, the resulting cationic SLP derivative can be extracted from the reaction mixture via a standard liquid-liquid extraction using an organic solvent, preferably ethyl acetate, washed with a pH 9.0 sodium carbonate buffer, and concentrated under reduced pressure (e.g., about 200 to 250 mbar, or about 240 mbar). The mixture can then be resuspended in deionized water and purified using cationic exchange resins.

In certain embodiments, the extracted cationic SLP are circulated through an ion exchange bed containing equimolar to 1.5 molar amounts of cation exchange sites to the concentration of the crude linear cationic SLP with, for example, a peristaltic pump or other type of pump, for a period of 2 to 20 hours, 3 to 15 hours, or 4 to 12 hours.

In preferred embodiments, removal of the SLP cationic derivatives from the resin is accomplished by application of an electrolyte solution containing a large concentration of monovalent metallic cations, wherein the large concentration is 1.5 to 15 molar equivalents, or 2 to 10 molar equivalents to the concentration of the SLP cationic derivatives. The monovalent metallic cations in large concentration outcompete the bound SLP cationic derivatives, allowing for them to exchange on the resin and produce a highly purified stream of SLP cationic derivatives.

In some embodiments, following installation of the cationic cargo, the resulting cationic SLP derivative can be purified by stirring it with saturated ammonium chloride solution to produce a stirred mixture; extracting the cationic SLP derivative by applying CH₂Cl₂ solvent (3×) to the stirred mixture to produce an extraction mixture; removing trace water from the extraction mixture by applying MgSO₄ or Na₂SO₄; drying the extraction mixture under elevated pressure (e.g., 350 to 450 mbar, or 400 mbar) and at 35 to 45° C. to remove the CH₂Cl₂ solvent; and, applying 21% NaOEt/EtOH solution, NaHCO₃ or KHCO₃ base in ethanol to remove acetyl R groups from the cationic SLP derivative. The de-acetylated linear cationic SLP derivative can then be converted to an HCl salt via reaction with a 1.25M HCl/EtOH solution.

### Formulations and Methods of Use

In some embodiments, the cationic surfactant molecules produced according to the subject methods can be used as active ingredients in environmentally-friendly, or “green,” cleaning compositions for efficiently disinfecting and/or sanitizing materials and/or surfaces contaminated with, for example, bacteria, viruses, fungi, molds, mildew, protozoa, biofilms, and/or other infectious organisms. Advantageously, in preferred embodiments, the compositions and methods are at least as effective for disinfecting and/or sanitizing materials and/or surfaces as antimicrobial peptides (AMPs), or cationic host defense peptides, as well as other chemical and/or synthetic cleaning formulations, such as QACs and SCAs.

Additionally, the cationic surfactants of the subject invention can be used in a wide range of cleaning applications due to their unique stability and efficacy across pH ranges. Thus, they enable disinfection as well as improved cleaning for broad categories of soils, including anionic soils such as anionic sugars and glycoproteins. This is particularly useful given that most pathogens of public health concern are typically transmitted via fomites, e.g., fecal soils, respiratory droplets, emesis, blood, other bodily fluids, and/or biofilms containing soils that protect the organisms against the action of common disinfectants.

As used herein, a “deleterious” or “pathogenic” microorganism refers to any single-celled or acellular organism that is capable of causing an infection, disease or other form of harm in another organism. As used herein, deleterious or pathogenic microorganisms are infectious agents and can include, for example, bacteria, cyanobacteria, biofilms, viruses, virions, viroids, fungi, molds, mildews, protozoa, prions, and algae.

As used herein, a “undesirable” microorganism refers to a non-pathogenic species whose growth on a surface or in a product can cause visible growth, odors, spoilage, or other organoleptic damage to a product. While not necessarily capable of causing an infection, such organisms can spoil foods, soil surfaces, create visible biofilms, and/or create undesirable odors. Their growth can make products unfit for use, especially foods, cosmetics, cleaning products, and personal care items. Non-limiting examples of undesirable microorganisms that cause spoilage or other physical but nonpathogenic changes to a product include certain species within the bacterial genera Lactobacillus, Pediococcus, Micrococcus, Streptococci, Propionibacterium, Streptomyces, Actinomycetes, and Bacilli, and fungal genera including Geotrichum, Penicillium, Saccharomyces, and Zygosaccharomyces.

As used herein, “contaminant” or “soil” refers to an impurity, or any substance that causes another substance or object to become fouled or impure. Contaminants can be living or non-living and can be inorganic or organic substances or deposits. Furthermore, contaminants can include, but are not limited to, hydrocarbons, such as petroleum or asphaltenes; fats, oils and greases (FOG), such as cooking grease, plant-based oils, and lard; lipids; waxes, such as paraffin; resins; microorganisms, such as bacteria, biofilms, viruses, fungi, molds, mildews, protozoa, parasites or another deleterious or undesirable microorganisms; stains; bodily fluids; plant matter; or any other substances referred to as, for example, dirt, dust, scale, sludge, crud, slag, grime, scum, schmoo, plaque, buildup, or residue.

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or move about in liquid medium.

As used herein, “fouling” means the accumulation or deposition of contaminants on a surface of, for example, a piece of equipment in such a way as to compromise the structural and/or functional integrity of the equipment. Fouling can cause clogging, plugging, deterioration, corrosion, and other problems associated therewith, and can occur on both metallic and non-metallic materials and/or surfaces. Fouling that occurs as a result of living organisms, for example, biofilms, is referred to as “biofouling.”

As used herein, “cleaning” means removal of undesirable soils, chemicals, or microbial contaminants from an unclean or fouled material and/or surface. Such contaminants may be undesirable because they indicate that a surface or material has been exposed to environmental soils, fluids, or microbes that make the surface visibly soiled, that make it unusable for its intended purpose, that may create undesirable odors, or that may harbor undesirable and/or deleterious microorganisms.

As used herein, to “preserve” means to prevent the growth of undesirable and/or deleterious microorganisms on a surface or in a material for a specific duration in time, minimally for at least 24 hours, preferably for up to 7 days, or most preferably for 30 days or longer, as measured from the time of treatment. In certain preferred embodiments, the materials to be preserved will be liquids, foods, cosmetic products, or personal care products.

As used herein, “preventing” a situation or occurrence refers to avoiding, delaying, forestalling, or minimizing the onset of a particular sign or symptom of situation or occurrence. Prevention can, but is not required to be, absolute or complete, meaning the situation or occurrence may still develop at a later time. Prevention can include reducing the severity of the onset of situation or occurrence, and/or inhibiting the progression of the situation or occurrence to one that is more severe.

As used herein, “control” refers to the physical or mechanical removal of contaminants such as soils and microbes. Control may include use of a preserving, sanitizing or disinfecting biocidal agent. “Residual control” further refers to pretreatment of a surface, fluid, or material with a process or composition that can kill, damage, or prevent the attachment, growth and survival of undesirable and/or deleterious microorganisms that may contact that surface or material sometime after treatment. Residual control treatments result in surfaces or materials that can resist subsequent colonization by undesirable and/or deleterious microorganisms.

As used herein, to “sanitize” means to inactivate or to kill at least 99.9% of undesirable or deleterious microorganisms on a surface or in a material within 10 minutes or less, preferably in 5 minutes or less, or most preferably in 2 minutes or less as measured from the time of contact between the composition and the microorganism (i.e., exposure time).

As used herein, to “disinfect” means to irreversibly inactivate or kill at least 99.999% of undesirable and/or deleterious microorganisms in 10 minutes or less, preferably in 5 minutes or less, most preferably in 2 minutes or less after the time of contact between the composition and the microorganism (i.e., exposure time).

In certain a preferred embodiment, at least 99.9% of each of two surrogates representing both Gram positive and Gram negative bacteria are killed in no more than 10 minutes, meaning the surface and/or material has been “sanitized.”

In the most preferred embodiment, at least 99.999% of specific surrogates representing both Gram positive and Gram negative bacteria are killed in no more than 10 minutes, meaning the surface and/or material has been “disinfected”.

“Surrogates” are strains that are recognized by regulatory authorities in the U.S., EU, or other countries as representing an entire class of microorganisms. In this case, Staphylococcus aureus is the most widely recognized surrogate for Gram positive bacteria, because it is considered to be the most difficult to disinfect or sanitize of all relevant Gram positive vegetative bacteria (with a few exclusions). In addition, either Salmonella enterica or Pseudomonas aeruginosa are typically considered to be the most difficult to either sanitize or disinfect of all relevant Gram negative bacteria.

The cleaning compositions of this invention can be formulated and delivered as liquids, suspensions, emulsions, dissolvable powders and/or granules, pressed powders, loose powders, diluted sprays, concentrates, aerosols, foams, encapsulated dissolvable pods, gels, and/or as a pre-moistened or water-activated cloth, sponge, wipe or other substrate. The cleaning compositions can be used as, for example, toilet bowl cleaners, laundry detergents, dishwashing detergents, hard and soft surface cleaners, water cleaners, air cleaners and/or carpet cleaners.

Certain preferred embodiments based on the inherent toxicological safety of these compositions enable creation of novel disinfecting agents that are skin contact safe, and that can be used in food preparation and storage areas, in the presence of children and pets, as preservatives in foods. Additional preferred embodiment includes the ability to disinfect surfaces in hospitals and nursing homes without the need to evacuate patients and without the requirements for Personal Protective Equipment (PPE) that current disinfecting actives such as hypochlorite and QACs require.

In some embodiments, the cationic surfactant derivative molecules produced according to the subject methods can be used as active ingredients in consumer products, serving as preservatives to prevent spoilage and/or growth of deleterious and/or undesirable organisms. Such consumer products can include, for example, cleaning products (e.g., disinfectants, all-purpose cleaners, glass cleaners, laundry and dish detergents), home care products (e.g., floor polish, air fresheners), personal care products (e.g., skin care products, hair care products), cosmetics (e.g., makeup, nail polish), painting and building supplies (e.g., paints, lacquers, primers, putty, drywall, caulk), and in some embodiments, health, food and beverage products.

Advantageously, the present invention eliminates environmental risks that are common to synthetically produced surfactants and disinfecting actives, such as QACs. The process for producing these molecules uses natural ingredients and soft processes that eliminate the dangerous gases and toxic compounds associated with QAC production, as well as reduce the risk of polluting water sources and interfering with waste water treatment processes. The molecules of the current invention are readily biodegradable and do not persist in the environment. Thus, the present invention can be used in a variety of industries as, e.g., a “green” disinfectant, sanitizer, preservative, cleaner and/or conditioner.

In certain embodiments, the cleaning composition and/or a consumer product comprising a cationic surfactant according to the subject invention, comprises the cationic surfactant at 0.1 to 10% by weight, 0.1 to 9.0%, 0.1 to 8.0%, 0.1 to 7.0%, 0.1 to 6.0%, 0.1 to 5.0%, 0.1 to 4.0%, 0.1 to 3.0%, 0.1 to 2.0%, 1.0 to 9.0%, 1.0 to 5.0%, 1.0 to 3.0%, 3.0 to 10%, 3.0 to 7.0%, 5.0 to 10%, 5.0 to 9.0%, 6.0 to 10%, 7.0 to 10% and 8.0 to 10%. In certain embodiments, the cationic surfactant is present in the composition at about 1 ppm to about 200 ppm, or about 2 ppm to about 250 ppm, or about 5 ppm to about 300 ppm, or about 10 ppm to about 350 ppm, or about 25 ppm to about 400 ppm, or about 50 ppm to about 450 ppm, or about 75 ppm to about 500 ppm, or about 100 ppm to about 600 ppm, or about 125 ppm to about 750 ppm, or about 150 ppm to about 1,000 ppm, or about 175 ppm to about 1,500 ppm, or about 0.5 ppm to about 2,000 ppm.

In a specific embodiment, the cationic surfactant is present at a concentration of 50 to 500 ppm of the cleaning composition or the consumer product. In another specific embodiment, the cationic surfactant is present at a concentration of 100 to 1,500 ppm.

In certain embodiments, the pH of the cleaning composition ranges from 2.0 to 12.0, 2.5 to 11, 3.0 to 10.0, 3.0 to 9.0 or 4.0 to 8.0. Known pH adjusters can be utilized in order to keep the pH at a suitable level, including, for example, hydrochloric acid, sulfuric acid, sodium carbonate or bicarbonate, sodium hydroxide, ammonium hydroxide, calcium hydroxide, magnesium hydroxide, acetic acid, lactic acid and/or citric acid.

Optionally, the cleaning composition can further comprise one or more other components, including, for example, carriers (e.g., water), other biosurfactants, other surfactants (e.g., polyalkyglucosides such as capryl glucoside and lauryl glucoside, amine oxides), hydrophilic and/or hydrophobic syndetics, sequestrants, builders (e.g., potassium carbonate, sodium hydroxide, glycerin, citric acid, lactic acid), solvents (e.g., water, ethanol, methanol, isopropanol), organic and/or inorganic acids (e.g., lactic acid, citric acid, acetic acid, boric acid), botanical extracts, cross-linking agents, chelators (e.g., potassium citrate, sodium citrate, sodium gluconate, citric acid, EDTA, DEDTA), fatty acids, alcohols, reducing agents, oxidants, calcium salts, carbonate salts, buffers, enzymes, dyes, colorants, fragrances (e.g., d-limonene, thymol, citral, lavender), preservatives (e.g., octylisothiazolinone, methylisothiazolinone), propellants, terpenes (e.g., d-limonene), sesquiterpenoids, terpenoids, emulsifiers, demulsifiers, foaming agents, defoamers, bleaching agents, polymers, thickeners and/or viscosifiers (e.g., xanthan gum, guar gum).

In an exemplary embodiment, the cleaning composition can comprise a cationic surfactant according to the subject invention formulated or delivered as a solution (1-50%) in a glycol solvent, such as, for example, glycerol, propylene, and/or butylene glycol. In certain embodiments, this exemplary formulation or delivery can further comprise up to 5% (relative to the active antimicrobial) of one or more acids such as, for example, acetic acid, lactic acid and/or citric acid.

In certain embodiments, the cleaning composition can comprise essential oils. Essential oils are volatile aromatic oils which may be synthetic or may be derived from plants by distillation, expression or extraction, and which usually carry the odor or flavor of the plant from which they are obtained. Useful essential oils may provide antiseptic activity. Some of these essential oils also act as flavoring agents. Useful essential oils include but are not limited to citra, thymol, menthol, methyl salicylate (wintergreen oil), eucalyptol, carvacrol, camphor, anethole, carvone, eugenol, isocugenol, limonene, osimen, n-decyl alcohol, citronel, α-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, tropolone derivatives such as hinokitiol, lavender oil, mustard oil, phenol, phenyl salicylate, pine oil, pine needle oil, sassafras oil, spike lavender oil, storax, thyme oil, tolu balsam, turpentine oil, clove oil, and combinations thereof.

In some embodiments, the composition comprises additional and/or other biosurfactants in crude and/or purified form. Additional biosurfactants according to the subject invention can include, for example, glycolipids, lipopeptides, flavolipids, phospholipids, fatty acid esters, and high-molecular-weight biopolymers such as lipoproteins, lipopolysaccharide-protein complexes, and/or polysaccharide-protein-fatty acid complexes.

In one embodiment, the additional and/or other biosurfactant is a glycolipid, such as, for example, rhamnolipids (RLP), cellobiose lipids, trehalose lipids and/or mannosylerythritol lipids (MEL). Natural (or non-derivatized) SLP can also be used. In one embodiment, the biosurfactant is a lipopeptide, such as, for example, surfactin, iturin, fengycin, arthrofactin, amphisin, viscosin, lichenysin, paenibacterin, polymyxin and/or battacin. In one embodiment, the biosurfactant is another type of amphiphilic molecule, such as, for example, esterified fatty acids, saponins, cardiolipins, pullulan, emulsan, lipomanan, alasan, and/or liposan.

In some embodiments, the biosurfactants are utilized in a crude form, wherein a biosurfactant molecule is present in the growth medium (e.g., broth) in which a biosurfactant-producing microorganism is cultivated and is collected therefrom without purification. The crude form can comprise, for example, at least 0.001%, 1%, 5%, 10%, 20%, 30%, 40%, 50%, 60%, 70%, 80%, 90% or 99% amphiphilic molecules in the growth medium. In alternate embodiments, the biosurfactant is extracted from the growth medium and, optionally, derivatized and/or purified.

In one embodiment, the biosurfactant is a biosurfactant alcohol ester, such as, for example, a lactonic sophorolipid ethyl ester, a lactonic sophorolipid methyl ester, a lactonic sophorolipid isopropyl ester, a lactonic sophorolipid butyl ester, a linear sophorolipid ethyl ester, a linear sophorolipid methyl ester, a linear sophorolipid isopropyl ester, or a linear sophorolipid butyl ester.

In one embodiment, the biosurfactant is a metal-biosurfactant complex, wherein an antimicrobial metal, such as silver, is added to the biosurfactant molecule. In certain embodiments, the complex is a silver-sophorolipid complex.

In one embodiment, the biosurfactant is a mixture of lipopeptide biosurfactants (e.g., surfactin, iturin, fengycin and/or lichenysin) produced by, for example, Bacillus amyloliquefaciens NRRL B-67928 or Bacillus subtilis NRRL B-68031. In certain embodiments, the mixture of lipopeptides comprises >50% surfactin.

Methods for Disinfecting, Sanitizing and/or Preserving

In preferred embodiments, the subject invention provides methods for disinfecting and/or sanitizing materials (including fluids, such as air and/or water), surfaces and/or fibers having an undesirable and/or deleterious microorganism therein or thereon, wherein the method comprises applying a cleaning composition according to the subject invention to the material, surface and/or fiber such that the composition is contacted with the microorganism.

In some embodiments, the subject invention provides methods for preserving a consumer product, wherein a cleaning composition and/or a cationic surfactant derivative according to the subject invention is formulated with the consumer product. Such consumer products can include, for example, cleaning products (e.g., disinfectants, all-purpose cleaners, glass cleaners, laundry and dish detergents), home care products (e.g., floor polish, air fresheners), personal care products (e.g., skin care products, hair care products), cosmetics (e.g., makeup, nail polish), painting and building supplies (e.g., paints, lacquers, primers, putty, drywall, caulk), and in some embodiments, pharmaceuticals, supplements, food and beverage products.

Advantageously, the methods are safe for use in household, commercial, and industrial settings and in the presence of humans, plants and animals.

The subject methods can be used to disinfect, sanitize and/or preserve from a broad spectrum of undesirable and/or deleterious microorganisms, including both Gram-negative and Gram-positive bacteria, biofilms, viruses (including enveloped viruses), fungi, molds, protozoa, parasites, algae, as well as other infectious organisms, such as worms and nematodes.

In certain specific embodiments, the methods can be used for disinfecting a material and/or surface having E. coli, Staphylococcus spp., Salmonella spp., Campylobacter spp., and/or Clostridium spp. thereon.

The cleaning composition can be applied to, for example, hard surfaces, soft porous surfaces, textiles and fibers, countertops, desks, floors, toilets, plastic, glass, ceramics, sinks, bathtubs, toys, doorknobs, carpets, rugs, windows, medical devices or implants, or fluids (e.g., air or water).

The cleaning composition can be applied directly to the material and/or surface by spraying using, for example, a spray bottle or a pressurized spraying device, or otherwise pouring or squeezing the composition onto or into the material and/or surface from a vessel. The cleaning composition can also be applied using a sponge, cloth, wipe or brush, wherein the composition is rubbed, spread or brushed onto the material and/or surface. Furthermore, the cleaning composition can be applied via a laundry washing machine or a dishwasher. Even further, the cleaning composition can be dispersed into air as an aerosol, which can be useful for disinfecting or removing airborne fomites, microorganisms and/or allergens.

The cleaning composition can be used independently from or in conjunction with an absorbent and/or adsorbent material. For instance, the cleaning composition can be formulated to be used in conjunction with a cleaning wipe, sponge (cellulose, synthetic, etc.), paper towel, napkin, cloth, towel, rag, mop head, squeegee, and/or other cleaning device that includes an absorbent and/or adsorbent material. The cleaning composition can be pre-loaded onto an absorbent and/or adsorbent material, post-absorbed and/or post adsorbed by a material during use, and/or be used separately from an absorbent and/or adsorbent material.

A cleaning wipe, upon which the improved cleaning composition can be loaded thereon, can be made of an absorbent/adsorbent material. Typically, the cleaning wipe has at least one layer of nonwoven material. Non-limiting examples of commercially available cleaning wipes that can be used include DuPont 8838, Dexter ZA, Dexter 10180, Dexter M10201, Dexter 8589, Ft. James 836, and Concert STD60LN. All these cleaning wipes include a blend of polyester and wood pulp. Dexter M10201 also includes rayon, a wood pulp derivative. The loading ratio of the cleaning composition onto the cleaning wipe can be about 2-5:1, or about 3-4:1. The cleaning composition is loaded onto the cleaning wipe in any number of manufacturing methods. Typically, the cleaning wipe is soaked in the cleaning composition for a period of time until the desired amount of loading is achieved. The cleaning wipe loaded with the improved cleaning composition provides excellent cleaning with little or no streaking/filming.

In one embodiment, the cleaning composition is left to soak on or in the material and/or surface for a sufficient time to achieve disinfection and/or sanitization. For example, soaking can occur for 5 seconds to 10 minutes, or from 10 seconds to 5 minutes, or from 30 seconds to 2 minutes. Preferably, the minimum exposure time required is less than 60 seconds, more preferably less than 30 seconds, in order to achieve disinfection and/or sanitization.

In one embodiment, the cleaning composition can be applied using agitation. This can be mechanical, for example, in a laundry washing machine or dishwasher, or manually, for example, by scrubbing with a cloth, wipe, sponge or brush.

In some instances, an undesirable and/or deleterious organism may be dried onto the surface or material to be cleaned, making it more difficult to solubilize or contact the organism with a disinfecting active ingredient. Furthermore, in some instances, a contaminated surface may be unclean, in addition to being contaminated with an undesirable and/or deleterious microorganism, meaning it also contains soils or other biological materials that make contact and disinfection of the undesirable and/or deleterious microorganism more difficult to achieve in the desired 10 minutes or less required for U.S. EPA or EU approved disinfection claims. Advantageously, however, the cleaning composition of the subject invention can be utilized as a “One Step Disinfectant,” as defined by the U.S. EPA, for cleaning and disinfecting a contaminated surface in a single application.

In some embodiments, the cleaning composition is left on the material or surface indefinitely and can provide preservation, disinfection and/or sanitization for, minimally, at least 24 hours, preferably for up to 7 days, more preferably for up to 14 days, or most preferably for 30 days or longer, as measured from the time of treatment.

In one embodiment, the method further comprises the step of removing the cleaning composition and undesirable and/or deleterious microorganism(s) from the material and/or surface. This can be achieved by, for example, rinsing or spraying water onto the surface, and/or rubbing or wiping the surface with a cloth, wipe, sponge or brush until the cleaning composition and microorganism(s) have been freed from the material and/or surface. Rinsing or spraying with water can be performed before, during and/or after rubbing or wiping the surface.

In some embodiments, methods for preventing spoilage or contamination of a consumer product are provided, wherein a cationic surfactant derivative according to the subject invention is applied with/to, or formulated with, the consumer product as a preservative ingredient. The consumer product can be, for example, a cleaning product, home care product, personal care product, cosmetic, painting and/or building supplies, and in some embodiments, a pharmaceutical, supplement, food and/or beverage product.

Target Microorganisms for Disinfecting, Sanitizing and/or Preservation

Advantageously, the methods can be used to disinfect, sanitize and/or preserve from a broad spectrum of undesirable and/or deleterious microorganisms, including both Gram-negative and Gram-positive bacteria, yeasts, molds, biofilms (including mixed species biofilms), enveloped viruses and non-enveloped viruses, mildews, and even algae.

In certain specific embodiments, the methods can be used for preserving, sanitizing, and/or disinfecting materials and/or surfaces that are prone to contamination with undesirable microorganisms therein or thereon, which include species and strains from Gram positive genera such as, for example, Bacillus, Alicyclobacillus, Geobacillus, Lactobacillus, Streptococci, Micrococcus, Pediococci, Leuconostoc, Oenococcus, Propionibacterium, Streptococcus, Enterococcus, Actinomyces, and Streptomyces;
species and strains of Gram negative genera such as, for example, Erwinia, Corynebacteria, Psychrobacter, Pseudomonas, Alcaligenes, Escherichia, Proteus, Serratia, Citrobacter, Aeromonas, Acinetobacter and Klebsiella; and
species of various fungal genera including, for example, Saccharomyces, Zygosaccharomyces, Geotrichum, Candida, and Penicillium.

A preferred embodiment of this invention enables the product of disinfectants, sanitizers, and preservatives that are safe and suitable for treatments of agricultural commodities, fresh and processed foods, cosmetics, personal care products, and cleaning products, as well as the facilities in which these products are made.

In another specific embodiment, the methods can be used for disinfection and/or sanitization of deleterious microorganisms known to cause disease or illness in humans, animals and/or plants. Deleterious organisms include life threatening foodborne and waterborne pathogens such as, for example, Campylobacter jejuni, Salmonella typhimurium, E coli 0157:H7, Staphylococcus aureus, Yersinia enterocolitica, Clostridium perfringens, Clostridium botulinum, Bacillus cereus, Bacillus subtilis, Escherichia coli, Xanthomonas campestris, Listeria monocytogenes, Enterococcus faecalis. Klebsiella pneumoniae, and Enterobacter aerogenes.

The subject invention can also assist in control and prevention of infectious pathogens of major public health concern, including, for example, bacteria such as Salmonella enterica, Salmonella choleraseus, Staphylococcus aureus (including MRSA), Staphylococcus saprophyticus, Streptococcus pyogenes, Streptococcus pneumoniae, Bacillus anthracis, Legionella pneumophila, Klebsiella pneumoniae, Shigella dysenteriae, Vibrio cholera, Vibrio parahaemolytics, vancomycin-resistant Enterococci, Mycobacterium tuberculosis, Mycobacterium bovis, Acinetobacter baumanii, Clostridium difficile;
fungi, such as, for example, Zygosaccharomyces spp., Debaryomyces hansenii, Candida spp., Dekkera/Brettanomyces spp., Leptosphaerulina chartarum, Epicoccum nigrum, Wallemia sebi, Cryptococcus spp., Trichophyton rubrum, Trichophyton mentagrophytes, Epidermophyton floccosum, including pathogens such as Candida albicans, Candida auris, and Mucor miehei;
molds, such as, for example, Alternaria, Aspergillus, Byssochlamys, Botrytis, Cladosporium, Fusarium, Geotrichum, Manoscus, Monilia, Mortierella, Mucor, Neurospora, Oidium, Oosproa, Penicillium; and
parasites, such as tapeworms, helminths, nematodes, Toxoplasma, Trichinella, Giardia lambila, Entamoeba histolytica, and Cryptospordium.

In certain embodiments, the cleaning composition can have disinfecting and/or sanitizing capabilities against enveloped and non-enveloped viruses, such as, for example, coronaviruses (including SARS-COV1 and CoV2), rotaviruses, norovirus, hepatitis A, B, and C, Coxsackievirus, Rhinovirus, the cold virus, the flu virus, herpes viruses, cytomegalovirus, and poliovirus.

### Conditioning Compositions and Methods of Use

The present invention provides conditioning compositions, as well as methods of their use in the conditioning of hair, fibers and textiles. More specifically, the present invention provides microbial-derived ingredients for use in formulating hair care products, household laundry products, and textile processing materials. Advantageously, in certain embodiments, the microbial-derived ingredients can be useful for replacing and/or reducing the use of traditional conditioning compounds, such as QACs and palm oil.

As used herein, the term “conditioning” refers to one or more of the following non-limiting examples as it is applicable to hair, fibers and/or textiles: reducing static and/or frizz; increasing strength; reducing breakage; reducing tangling and matting; improving shine and softness; increasing oil and/or moisture retention; reducing wrinkles; and/or imparting a fragrance.

In some embodiments, the conditioning compositions can be used in a hair care product, such as, for example, a shampoo, a conditioner or cream rinse, a de-tangler, or a styling product. In some embodiments, the conditioning composition can be used in a household or industrial laundry product, such as a fabric softener or a dryer sheet. In some embodiments, the conditioning composition can be used in a treatment for the conditioning of raw fibers or fabrics prior to the assembly of garments, fabrics, rugs and other textiles.

In preferred embodiments, the conditioning composition comprises one or more cationic surfactants according to the subject invention. The cationic surfactants can be included at a dosing rate of about 100 ppm to about 250,000 ppm, about 200 ppm to about 100,000 ppm, about 300 ppm to about 75,000 ppm, about 400 ppm to about 50,000 ppm, or about 500 ppm to about 25,000 ppm.

In certain embodiments, the conditioning composition comprises the cationic surfactants at of dosing rate of about 0.01 wt % to about 25 wt %, about 0.05 wt % to about 20 wt %, about 0.1 wt % to about 15 wt %, or about 0.5 wt % to about 10 wt %.

In certain specific embodiments, the conditioning composition can further comprise additional biosurfactants. For example, in some embodiments, the conditioning composition comprises a linear cationic SLP derivative and one or more other SLP molecules that have not been derivatized according to the present invention.

In certain embodiments, the one or more biosurfactants include glycolipids selected from, for example, sophorolipids (SLP), mannosylerythritol lipids (MEL), rhamnolipids (RLP) and trehalose lipids (TL). The biosurfactants can be derivatized or in their natural state.

In some embodiments, the biosurfactants are utilized in a crude form, wherein a biosurfactant molecule is present in the growth medium (e.g., broth) in which a biosurfactant-producing microorganism is cultivated and is collected therefrom without purification. The crude form can comprise, for example, at least 0.001%, 1%, 5%, 10%, 20%, 30%, 40%, 50%, 60%, 70%, 80%, 90% or 99% amphiphilic molecules in the growth medium. In alternate embodiments, the biosurfactant is extracted from the growth medium and, optionally, derivatized and/or purified.

The mixture ratio of the cationic SLP derivative to the non-derivatized SLP can range from 1:1 to 1:1000, from 1:5 to 1:500, or from 1:10 to 1:100.

In other embodiments, the conditioning composition comprises a linear cationic SLP derivative and one or more mannosylerythritol lipid (MEL) molecules.

The mixture ratio of the cationic SLP derivative to the MEL can range from 1:1 to 1:1000, from 1:5 to 1:500, from 1:10 to 1:100, from 1000:1 to 1:1, from 500:1 to 5:1, or from 100:1 to 10:1.

In yet another exemplary embodiment, the conditioning composition comprises a derivatized linear cationic SLP, one or more other SLP molecules, and one or more MEL molecules.

In one embodiment, MEL comprise either 4-O-B-D-mannopyranosyl-meso-erythritol or 1-O-B-D-mannopyranosyl-meso-erythritol as the hydrophilic moiety, and fatty acid groups and/or acetyl groups as the hydrophobic moiety. One or two of the hydroxyls, typically at the C4 and/or C6 of the mannose residue, can be acetylated. Furthermore, there can be one to three esterified fatty acids, from 8 to 12 carbons or more in chain length.

MEL and MEL-like substances (e.g., mannose-based substances) are produced mainly by Pseudozyma spp. (e.g., P. aphidis) and Ustilago spp. (e.g., U. maydis), with significant variability among MEI, structures produced by each species. Certain mannose-based substances having similar properties to MEL can also be produced by Meyerozyma guilliermondii yeasts.

MEL are non-toxic and are stable at wide temperature and pH ranges. Furthermore, MEL can be used without any additional preservatives.

MEL can be produced in more than 93 different combinations that fall under 5 main categories: MEL A, MEL B, MEL D, Tri-acetylated MEL A, and Tri-acetylated MEL B/C. These molecules can be modified, either synthetically or in nature. For example, MEL can comprise different carbon-length chains or different numbers of acetyl and/or fatty acid groups.

MEL molecules and/or modified forms thereof according to the subject invention can include, for example, tri-acylated, di-acylated, mono-acylated, tri-acetylated, di-acetylated, mono-acetylated and non-acetylated MEL, as well as stereoisomers and/or constitutional isomers thereof.

Other mannose-based substances/MEL-like substances that exhibit similar structures and similar properties, can also be used according to the subject invention, e.g., mannosyl-mannitol lipids (MML), mannosyl-arabitol lipids (MAL), and/or mannosyl-ribitol lipids (MRL).

In certain embodiments, the composition comprises a carrier. Non-limiting examples of carriers may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, preferably organomodified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 1% to about 99% by weight of the composition, from 10% to about 85%, from 25% to 75%, or from 50% to about 65%.

Optionally, the conditioning composition can further comprise one or more other components as relevant to the specific use, including, for example, organic and/or inorganic solvents, organic and/or inorganic acids, essential oils, botanical extracts, cross-linking agents, chelators, fatty acids, alcohols, pH adjusting agents, reducing agents, buffers, enzymes, dyes, colorants, fragrances, preservatives, emulsifiers, demulsifiers, foaming agents, defoamers, bleaching agents, emollients, humectants, anti-inflammatory agents, polymers, stabilizers, silicones, thickeners, softeners, UV blockers, moisturizers, film formers, minerals, vitamins, proteins, viscosity and/or rheology modifiers, insect repellents, skin cooling compounds, skin protectants, lubricants, pearls, chromalites, micas, anti-allergenics, antimicrobials (e.g., antifungals, antivirals, antibacterials), antiseptics, pharmaceutical agents, photostabilizing agents, surface smoothers, optical diffusers, exfoliation promoters, anti-static agents, anti-wrinkling agents, wetting agents, dye transfer aids, color protectants, anti-odorants, odor capturing agents, detergents, drying agents, water repellency agents, anti-pilling agents, souring agents, starch agents, optical brightness agents, antioxidants, shrinkage control agents, starches, and mixtures thereof.

The amounts of each ingredient, whether active or inactive, are those conventionally used in cosmetics/personal care, textile processing and laundry care, to achieve their intended purpose, and typically range from about 0.0001% to about 25%, or from about 0.001% to about 20% of the composition, although the amounts may fall outside of these ranges. The nature of these ingredients and their amounts must be compatible with the production and function of the compositions of the disclosure. In preferred embodiments, the composition comprises additives that are considered dermatologically-acceptable.

As used herein, “dermatologically-acceptable,” “cosmetically-acceptable” and “topically-acceptable” are used interchangeably and are intended to mean that a particular component is safe and non-toxic for application to the integument (e.g., skin, scalp) at the levels employed. In one embodiment, the components of the composition are recognized as being Generally Regarded as Safe (GRAS).

In certain embodiments, the composition can include pH adjusters (e.g., citric acid, ethanolamine, sodium hydroxide, etc.) to be formulated within a wide range of PH levels. In one embodiment, the pH of the conditioning composition ranges from 1.0 to 13.0. In some embodiments, the pH of the conditioning composition ranges from 2.0 to 12.0. In some embodiments, the pH of the composition is from 3.0 to 7.0 or from 3.0 to 8.0.

The present invention can take any number of forms. These can include, for example, liquid; colloidal dispersion; micro- or nano-emulsion; gel; serum; granular, spray-dried or dry-blended powder; solid bar; concentrate; encapsulated dissolvable pod; suspension; hydrogel; multiphase solution; vesicular dispersion; foam; mousse; spray; aerosol; liquid cake; ointment; essence; paste; tablet; water soluble sheets or sachets; and/or can be impregnated into a dry or pre-moistened substrate such as a sheet (e.g., dryer sheet), ball (e.g., wool dryer ball), cloth, sponge or wipe.

### Methods of Conditioning Hair, Fibers and Textiles

In preferred embodiments, the subject invention further provides methods for conditioning hair, fibers or textiles, wherein the method comprises contacting a conditioning composition of the present invention with the hair, fibers or textiles for an effective amount of time to impart a conditioning effect thereon. In some embodiments, the composition is applied in the presence of water or another solvent. In some embodiments, the composition is rinsed from the hair, fibers or textiles after being contacted therewith.

In the context of a hair conditioner, the methods can comprise contacting the hair with the composition. In some embodiments, the composition is contacted with the hair while the hair is wet, while in other embodiments, the hair is dry. Application can comprise lathering or rubbing the composition into the strands of the hair; spraying the composition onto the hair; combing the composition through the hair; and/or other standard modes of application for hair care products.

In certain embodiments, the composition is applied to the hair simultaneously with shampooing or after shampooing the hair (i.e., after cleansing and rinsing the hair). The conditioning composition can be left in the hair as a leave-in conditioning treatment, or the composition can be rinsed after, for example, at least 15 seconds, at least 30 seconds, at least 1 minute, at least 5 minutes, to at least 60 minutes of contact.

In the context of a fiber conditioner, the composition can be contacted with fibers that are utilized in the assembly of garments and other textiles. In certain embodiments, the fibers are contacted with the composition prior to being assembled into textiles.

Chemical surfactants are often used to scour, or clean, raw fibers prior to further processing. Because scouring can be drying to the fibers, conditioning or lubrication is necessary prior to spinning, cording, and weaving the fibers into textiles. Thus, in certain embodiments, the conditioning composition can be contacted with the fibers to lubricate and soften the fibers, thereby reducing breakage, dryness, static and/or stiffness after scouring.

In the context of textile conditioning, the subject methods can be utilized during the finishing stages of textile manufacturing, as well as in household and industrial laundry. For example, in some embodiments, the conditioning composition is utilized in the form of a fabric softener that is applied to garments and other textiles during a standard wash cycle, during a drying cycle, or as a spray to wet or dry textiles.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Cationic Amino Alcohol Surfactant

In certain embodiments, the derivative surfactant compound is a cationic amino alcohol surfactant with particular utility as a broad-spectrum cleaning composition, disinfectant and/or a conditioner for hair/textiles. Additionally, these surfactants bear a primary or tertiary alcohol that can further be alkoxylated to increase molecular weight.

The cationic amino alcohol surfactant can be produced using any combination of acyl halides with a cationic amino acid alkyl ester reagent, such as, e.g., those depicted in Table 1.

TABLE 1
Exemplary reagents X′, Y′ and Z′ for producing
a cationic derivative surfactant compound.

| X′ | Y′ | Z′ |
|---|---|---|
| Lauroyl chloride | Lysine ethyl ester | LAH |
| Octanoyl chloride | Arginine ethyl ester | MeMgBr |
| Decanoyl chloride | Homoarginine ethyl ester | EtMgBr |
| Dodecanoyl chloride | Histidine ethyl ester | MePhMgBr |
| Myristyroyl chloride | | PhMgBr |
| Palmitoyl chloride | | nBuLi |
| Behenoyl chloride | | secBuLi |
| Long chain sophorolipid | | |
| Short chain sophorolipid | | |

LAH = lithium aluminium hydride;
MeMgBr = methyl magnesium bromide;
EtMgBr = ethyl magnesium bromide;
MePhMgBr = methyl phenylmagnesium bromide;
PhMgBr = phenylmagnesium bromide;
nBuLi = n-Butyllithium;
secBuLi = sec-Butyllithium

The following exemplary formula is produced using X′=dodecanoyl (lauroyl) chloride, Y′=arginine ethyl ester, Z′=LAH:

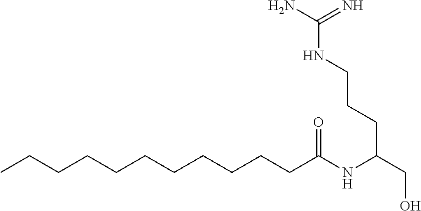 [SMILES: CCCCCCCCCCCC(=O)NC(CO)CCCNC(=N)N]

### Example 2—Microbial, Kill Study Protocol

Studies were conducted to determine the antimicrobial capabilities of cationic arginine derivatized surfactants produced according to embodiments of the subject invention.

The test organisms were a mixture of Gram-negative and Gram-positive organisms, which served to validate the breadth of the spectrum of control and the utility of the technology for common pathogens of public health concern: Pseudomonas aeruginosa (Gram-negative), Staphylococcus aureus (Gram-positive) and Salmonella entericus (Gram-negative).

Test bacteria were grown at 32° C. for 24-48 hours in tryptic soy broth, then plated at a starting concentration of 10⁷. PBS was used for dilution to achieve the desired cell concentration.

A stock solution of each antimicrobial treatment was created in water, which was then used to create each desired dilution for treatment: 25, 50, 200, 400, 500, 750, 1,000, 1,500, 2,000 and 4,000 ppm, in an appropriate volume of buffer. Buffers included 100 mM of citric acid, ammonium HCL, PBS or sodium bicarbonate.

The created cell dilution of 10⁷ CFU/mL was mixed with either 100 uL to 900 uL of antimicrobial at the appropriate PPM, or 1-9 mL of antimicrobial at the appropriate PPM. The final concentration of the organism was 106 once mixed with the antimicrobial.

The moment the organism was added to the antimicrobial, the timer was started for the desired time period (0, 2, 5, 6 or 10 min). At the end of the timer, the antimicrobial was neutralized, and dilutions were then plated onto TSA plates and incubated for 24-48 hours at 32° C.

The negative control comprised 0.1 mL from the organism stock plated onto a TSA plate. For the positive control a serial dilution was set up with a starting concentration of 400 PPM quaternary ammonium compound (QAC) and organism. Dilutions were plated at 10², 10³ and 10⁴ CFU/mL.

After 24 to 48 hours of incubation, plates were removed from the incubator and colony numbers were counted.

The below scale is commonly used in determining a compound's efficacy, where a greater than or equal to 6-fold logarithmic (≥6 log) reduction in less than 10 minutes is needed for disinfection.

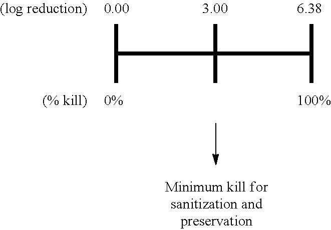

### Example 3—Surface Tension Reduction and Log Reduction Comparisons

Table 2 summarizes surface tension reduction at 1000 ppm and log reduction of P. aeruginosa at 0 and 10 min. (200 ppm) for two C12 cationic amino alcohol surfactants according to embodiments of the subject invention, LRO (R) and LKO (K), where LRO was produced through the coupling of a lauroyl chloride substrate and arginine ethyl ester and LKO was produced through the coupling of a lauroyl chloride substrate and lysine ethyl ester.

TABLE 2
Surface tension reduction and log reduction
of Pseudomonas aeruginosa (G−)

| | |
|---|---|
| Surface | |
| Tension | Log Reduction |
| 1000 ppm | 200 ppm |

| X | Y | (mN/m) | 0 min | 10 min |
|---|---|---|---|---|
| C12 | R | 23.75 | 1.85 | 6.25 |
| C12 | K | 27.82 | 0.54 | 6.25 |

Table 3 summarizes surface tension reduction at 1000 ppm and log reduction of S. aureus and S. enterica at 0 and 10 min. (200 ppm) for four categories of cationic amino alcohol surfactants according to embodiments of the subject invention.

The surfactants were produced through the coupling of octanoyl chloride (C8); decanoyl chloride (C10); lauroyl chloride (C12); myristyroyl chloride (C14); palmitoyl chloride (C16); or a linear sophorolipid (SLP), with: arginine ethyl ester (R), histidine ethyl ester (H) or lysine ethyl ester (K).

TABLE 3
Surface tension reduction and log reduction of Staphylococcus
aureus (G⁺) and Salmonella enterica (G⁻)

| | | | Surface | Log Reduction | | Log Reduction | |
|---|---|---|---|---|---|---|---|
| | | | Tension | S. aureus (G⁺) | | S. enterica (G⁻) | |
| | | | 1000 ppm | 200 ppm | | 200 ppm | |
| X | Y | Z | (mN/m) | 0 min | 10 min | 0 min | 10 min |
| C8 | R | O | 32.15 | 0.00 | 0.87 | 0.00 | 0.00 |
| C10 | R | O | 28.30 | 0.00 | 6.30 | 0.00 | 6.30 |
| C12 | R | O | 23.75 | 0.00 | 6.24 | 0.00 | 6.20 |
| C14 | R | O | 39.54 | 0.00 | 6.36 | 0.00 | 6.11 |
| C16 | R | O | 37.63 | 0.00 | 4.10 | 0.00 | 2.86 |
| SLP | R | O | 41.89 | 0.00 | 2.23 | 0.00 | 0.00 |
| C8 | H | O | 57.78 | 0.00 | 0.00 | 0.00 | 0.00 |
| C10 | H | O | 50.30 | 0.00 | 0.00 | 0.00 | 0.00 |
| C12 | H | O | 27.36 | 0.00 | 0.00 | 0.00 | 0.00 |
| C14 | H | O | 22.77 | 0.00 | 0.00 | 0.00 | 0.00 |
| C12 | K | O | 27.82 | 0.00 | 4.60 | 0.00 | 6.16 |

### Example 4—Stability Study

Tables 4-5 summarize the pH stability over 4 days for a C12 cationic amino alcohol surfactant according to embodiments of the subject invention, LRO (RO), compared with LAE (RE), where LRO was produced through the coupling of a lauroyl chloride substrate and arginine ethyl ester. Log reductions were measured according to the procedures described above in Example 2. Percent reduction in activity for both LRO and LAE at pH 7 is shown in Table 6.

TABLE 4
Day 0 Stability

| | | | | Log Reduction | | | Log Reduction | | |
|---|---|---|---|---|---|---|---|---|---|
| | | | | S. aureus (G⁺) | | | S. enterica (G⁻) | | |
| | | | | 100 ppm | | | 100 ppm | | |
| X | Y | Z | pH | 0 min | 2 min | 5 min | 0 min | 2 min | 5 min |
| C12 | R | O | 7 | 0.00 | 2.17 | 6.53 | 0.00 | 1.32 | 2.36 |
| C12 | R | E | 7 | 0.00 | 6.53 | 6.53 | 0.00 | 6.42 | 6.42 |

TABLE 5
Day 4 Accelerated Stability

| | | | | Log Reduction | | | Log Reduction | | |
|---|---|---|---|---|---|---|---|---|---|
| | | | | S. aureus (G⁺) | | | S. enterica (G⁻) | | |
| | | | | 100 ppm | | | 100 ppm | | |
| X | Y | Z | pH | 0 min | 2 min | 5 min | 0 min | 2 min | 5 min |
| C12 | R | O | 7 | 0.00 | 0.76 | 6.53 | 0.00 | 0.00 | 0.79 |
| C12 | R | E | 7 | 0.00 | 0.93 | 1.14 | 0.00 | 0.00 | 0.00 |

TABLE 6
Percent activity reduction

| | | | | Activity | Activity |
|---|---|---|---|---|---|
| | | | | Reduction S. | Reduction S. |
| | | | | aureus (G⁺) | enterica (G⁻) |
| | | | | 100 ppm | 100 ppm |
| X | Y | Z | pH | 5 min | 5 min |
| C12 | R | O | 7 | 0.00 | 66.53 |
| C12 | R | E | 7 | 82.54 | 100.00 |

### REFERENCES

Czakaj. A. et al. (2021). Ethyl Lauroyl Arginate, an Inherently Multicomponent Surfactant System. Molecules. 26, 5894. https://doi.org/10.3390/molecules26195894 (“Czakaj et al. 2021”).

1. A method for producing a cationic sophorolipid (SLP) derivative, the method comprising:
a) producing a linear SLP molecule comprising a carboxylic acid fatty acid tail, and
b) installing an amide comprising one or more cationic amino acid alkyl ester functional groups to the carboxylic acid tail of the linear SLP molecule to produce a long-chain amide.

2-4. (canceled)

5. The method of claim 1, wherein the SLP is a linear SLP molecule with a C18 carboxylic acid tail comprising a single unsaturation, and wherein the method comprises using oxidative cleavage to produce a carboxylic acid tail truncated at the ninth position and installing an amide comprising one or more amino acid-derived functional groups to the truncated carboxylic acid tail to produce a short-chain SLP amide.

6. The method of claim 1, wherein the amide comprises an amino alcohol.

7-8. (canceled)

9. A cationic surfactant having a structure selected from: 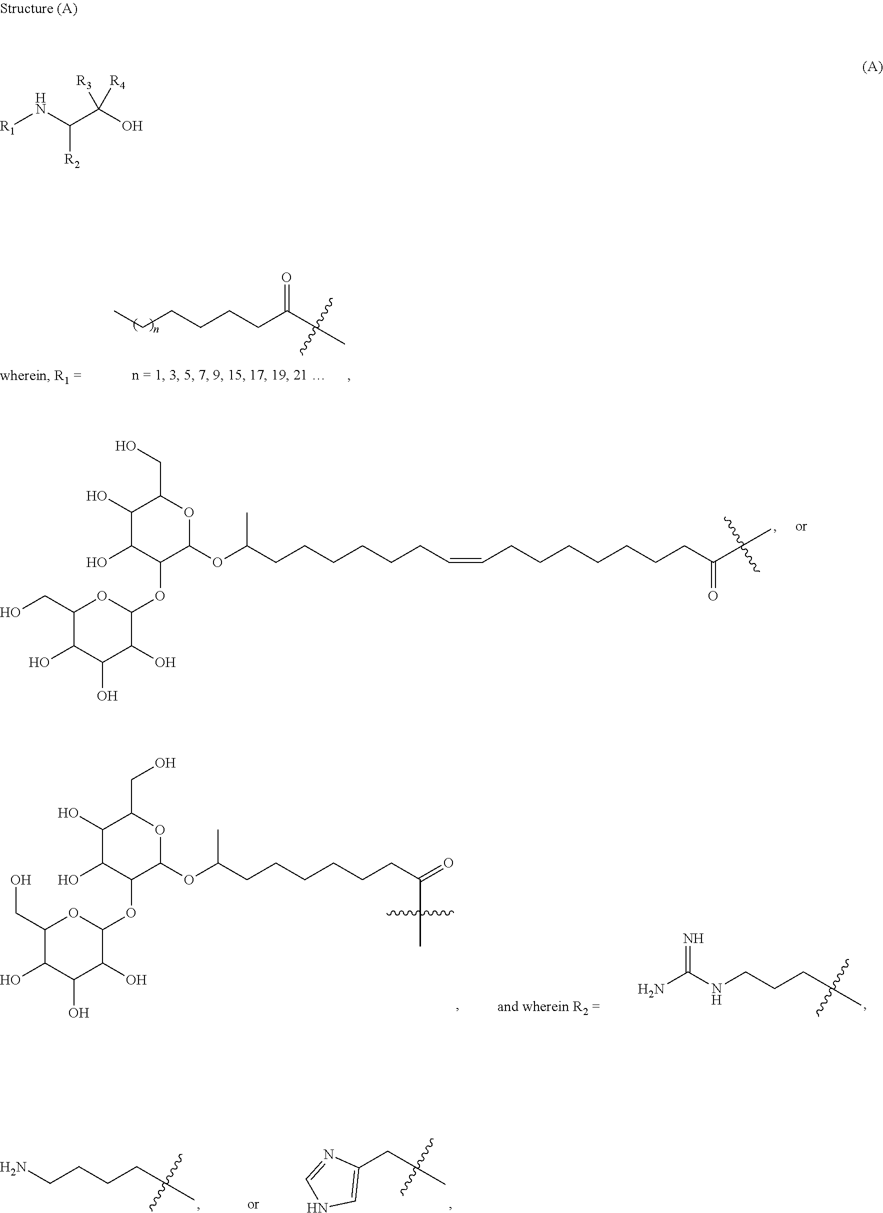
and wherein R₃=H, Mc, Et, MePh, Bu, sec-Bu, t-Bu; 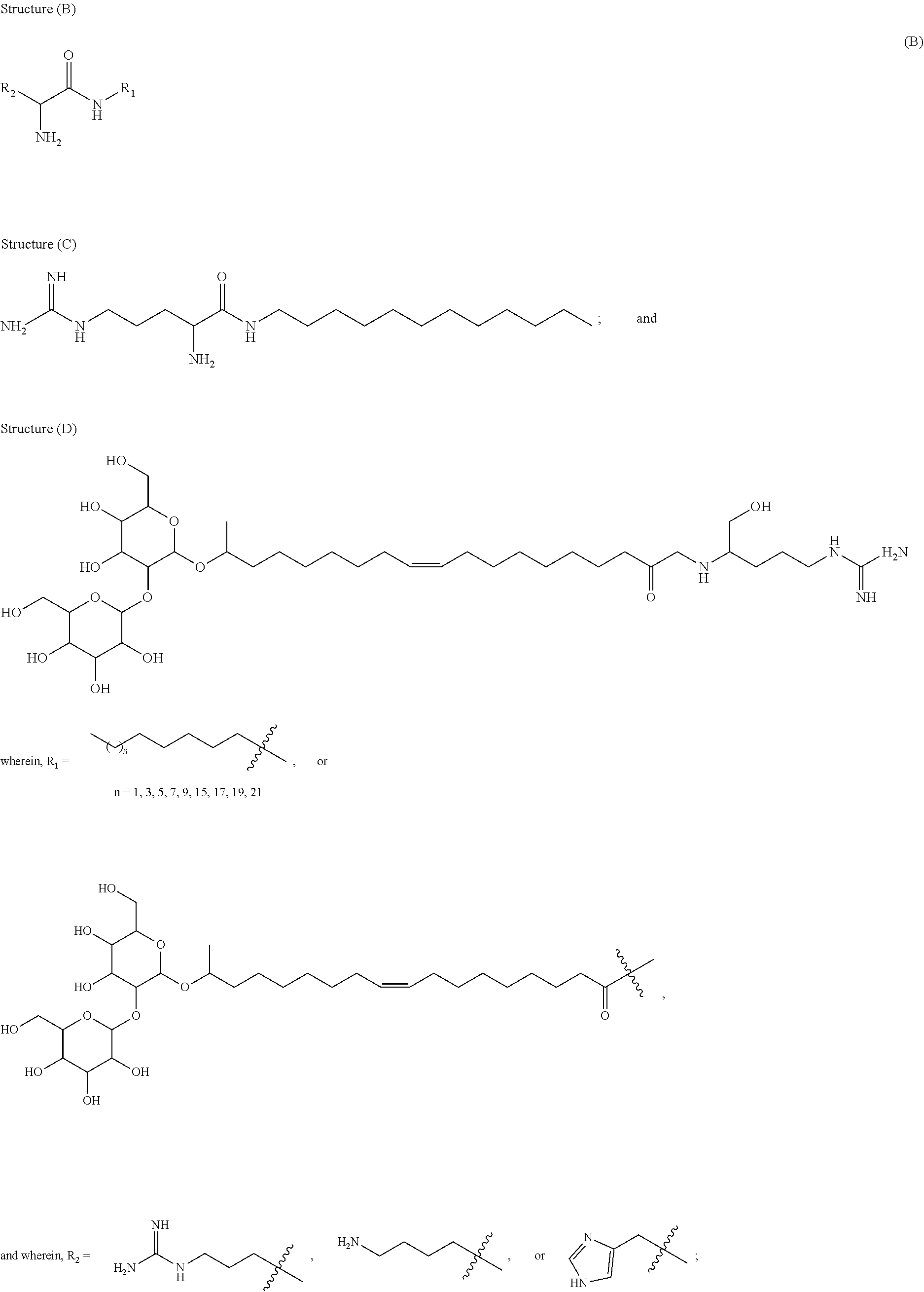
wherein structure (C) is a lauryl arginine amide and structure (D) is a linear sophorolipid argininol amide.

10. The cationic surfactant of claim 9, having one of the following structures: 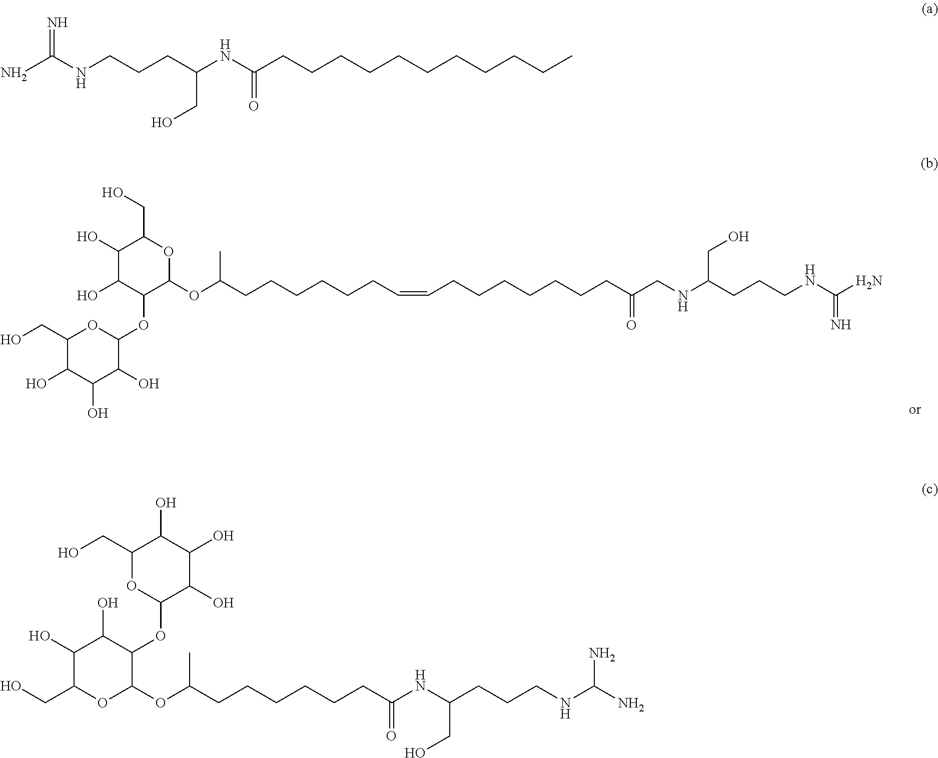 [SMILES: CCCCCCCCCCCC(=O)NC(CO)CCCNC(=N)N ; CC(CCCCCC/C=C\CCCCCCCC(=O)CNC(CO)CCCNC(=N)N)OC1OC(CO)C(O)C(O)C1OC1OC(CO)C(O)C(O)C1O ; CC(CCCCCCC(=O)NC(CO)CCCNC(N)N)OC1OC(CO)C(O)C(O)C1OC1OC(CO)C(O)C(O)C1O]
wherein structure (a) is a lauryl argininol alcohol, structure (b) is a linear sophorolipid argininol amide, and structure (c) is a truncated sophorolipid argininol amide.

11-12. (canceled)

13. A cleaning composition comprising a cationic surfactant according to claim 9, wherein the cleaning composition has pH 2-12.

14-16. (canceled)

17. The cleaning composition of claim 13, comprising a 1-50% solution of the cationic surfactant in a glycol solvent selected from glycerol, propylene and butylene glycol.

18-20. (canceled)

21. A method of disinfecting, sanitizing and/or preserving a material and/or surface that is contaminated with an undesirable or deleterious microorganism, the method comprising applying a cleaning composition of claim 13 to the material and/or surface such that the composition is contacted with the microorganism.

22-38. (canceled)

39. A method for purifying a sophorolipid (SLP), the method comprising circulating a crude SLP through an ion exchange bed containing ion exchange sites for a period of time from 30 minutes to 3 hours.

40-42. (canceled)

43. The method of claim 39, used for quenching a reaction involving a sophorolipid molecule.

44. A conditioning composition comprising a cationic surfactant molecule of claim 9, wherein the conditioning composition has a pH of 2-12.

45-46. (canceled)

47. The composition of claim 44 further comprising a mannosylerythritol lipid (MEL).

48-51. (canceled)

52. A method of conditioning hair, a fiber or a textile, the method comprising contacting a composition of claim 44 with the hair, fiber or textile.

53-54. (canceled)

55. A method for improving the pH stability of a molecule comprising an ester moiety, the method comprising converting the ester moiety to an alcohol, wherein the molecule is an amino acid ethyl ester, and wherein the method comprises converting the amino acid ethyl ester to an amino alcohol.

56. (canceled)

57. The method of claim 55, wherein the molecule is lauryl arginine ethyl ester (LAE) and the method comprises converting the LAE to a lauryl amino alcohol.

58. (canceled)

59. A method for producing a cationic surfactant compound with stability at pH 2-12, said compound having the following structure: 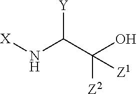
wherein X is a C2-C22 fatty amide derived from a fatty acid or a biosurfactant comprising a fatty acid moiety, Y comprises an amino acid-derived functional group, and Z¹ and Z² are independently a hydrogen or alkyl group,
the method comprising coupling a biosurfactant or fatty acid-containing substrate X′ with an amide Y′ comprising one or more amino acid side chains to produce an amino-acid functionalized surfactant XY, and
either before, during or after coupling of XY, reacting Y′ or XY with a reducing agent Z′ to reduce any ester groups existing therein to an alcohol.

60. The method of claim 59, wherein X′ is an acyl halide.

61. The method of claim 60, wherein the acyl halide is lauroyl chloride, octanoyl chloride, decanoyl chloride, dodecanoyl chloride, myristyroyl chloride, palmitoyl chloride or behenoyl chloride.

62. The method of claim 59, wherein Y comprises a side chain of an amino acid selected from lysine, arginine, homoarginine and histidine.

63. The method of claim 59, wherein X′ is a sophorolipid (SLP) biosurfactant.

64-65. (canceled)
